(19) United States
(12) Patent Application Publication
(10) Pub. No.: US 2019/0161756 A1
(43) Pub. Date: May 30, 2019

(54) Anti-Immunoglobulin G Aptamers and Uses Thereof

(71) Applicant: Laboratoire Francais du Fractionnement et des Biotechnologies, Les Ulis (FR)

(72) Inventor: Alexander SEIFERT, Paris (FR)

(73) Assignee: Laboratoire Francais du Fractionnement et des Biotechnologies, Les Ulis (FR)

(21) Appl. No.: 16/320,602

(22) PCT Filed: Jul. 6, 2017
(86) PCT No.: PCT/EP2017/066945

§ 371 Date: Jan. 25, 2019

(30) Foreign Application Priority Data
Jul. 28, 2016 (EP) 16305983.5
Jul. 28, 2016 (EP) 16305985.0

(51) Int. Cl. C12N 15/115; C07K 1/22; C07K 14/75

(52) U.S. Cl. CPC C12N 15/115; C07K 1/22; C12N 2320/52; C12N 2310/16; C07K 14/75

(57) ABSTRACT

The invention relates to aptamers which specifically bind to immunoglobulin G and their use in the purification of said protein.

### FIELD OF THE INVENTION

The invention relates to affinity ligands which specifically bind to immunoglobulin G (IgG) and their use in the purification of said protein.

### BACKGROUND OF THE INVENTION

Immunoglobulin G (IgG), which is a major protein of serum, plays an important role in the immune system by recognizing and eliminating foreign matter. In healthy adults, the four polypeptide chain IgG monomer constitutes approximately 75% of total serum immunoglobulins. IgG has a Y-shaped structure wherein two H chains and two L chains are bound via disulfide bonds (S—S bonds). When decomposed with the proteinase papain, IgG can be divided into an Fc fragment, which consists of a constant region; and a Fab fragment, which comprises an antigen-binding site. Human IgG has been subdivided into four subclasses on the basis of unique antigenic determinants Relative subclass percentages of total IgG in serum are IgG1, 50-70%; IgG2, 20-40%; IgG3, 2-10%; and IgG4, 1-8%. IgG1, IgG2 and IgG4 possess a molecular weight of approximately 150,000, whereas IgG3 is heavier (160,000 molecular weight).

IgG is widely studied for applications to therapeutic drugs, diagnostic reagents for various diseases, and test reagents. Such applications include antibody therapies for cancer; therapies based on antibody-dependent cellular cytotoxicity (ADCC) or complement-dependent cytotoxicity (CDC). IgG is also used as an essential tool for a range of biochemical experiments on the basis of its property of specific binding to antigens, for example cell or protein functional analysis and immunoassay.

Each therapeutic antibody can be used alone, in combination with chemotherapy, or as a carrier for toxins or radiation. Recent advancements in relevant technologies have facilitated the development of monoclonal antibody therapies. During purification of therapeutic antibodies, impurities, including host cell proteins, DNA, antibody variants, and small molecules, must be removed. Since many of the monoclonal antibody therapies require high doses and/or continued administration, economical and quality-controlled large-scale production of these antibodies is of great importance.

The common procedure used in purification of antibodies is protein A affinity chromatography because it efficiently and selectively binds to antibodies in complex solutions, such as harvested cell culture media. Protein A, which is a natural product of Staphylococcus aureus, binds to the Fc portion of a variety of mammalian IgG molecules. The main disadvantages of protein A chromatography include cost, quality control difficulties, resin stability, and acidic elution procedures which can impair the antibody's conformation and activity. Moreover, protein A is obtained from genetically modified bacteria through complex and expensive procedures explaining why protein A resin is over 30 times more expensive than other ion exchange resins, and may account for >35% of the total raw material costs for largescale recovery of IgG. Also, since protein A molecules may cause immunogenic or other physiological responses in humans, any contaminating ligand leaked from the base matrix must be removed during processing.

To overcome these disadvantages, several synthetic ligands have been proposed as replacements for protein A in the affinity purification of antibodies; these include the use of a thiophilic ligand, histidyl ligand, Avid A1, or peptides or nonpeptides designed to mimic protein A. However, none of these have yet become protein A alternatives at the manufacturing level.

The production of IgG in milk of transgenic animals and its subsequent purification has been also described, for instance in PCT applications WO9517085 and WO9419935. However, the IgG purification from milk is still a real challenge because the final product must be devoid of any non-human contaminating proteins which may be antigenic.

There is thus a need for alternative methods for the purification for IgG.

### SUMMARY OF THE INVENTION

The invention relates to an aptamer which specifically binds to at least 2 different subclasses of IgG selected from human IgG1, IgG2, IgG3 and IgG4. In some embodiments, the aptamer specifically binds to IgG1, IgG2, IgG3 and IgG4. In some additional or alternative embodiments, the aptamer binds the IgG in a pH-dependent manner. In some further embodiments, the aptamer of the invention is directed against the Fc domain of a IgG.

The invention also relates to an aptamer capable of specifically binding to IgG which comprises a moiety selected from the group consisting of SEQ ID N°16, SEQ ID N°17 and SEQ ID N°18, or which differs from a moiety selected from the group of SEQ ID N°16, SEQ ID N°17, and SEQ ID NO: 18 in virtue of 1, 2, 3, 4, or 5 nucleotide modifications.

In some embodiment, the aptamer of the invention comprises a polynucleotide:
having at least 70%, of identity with a sequence selected from the group of SEQ ID NO:1-15, and SEQ ID NO:21-23, and
comprising a moiety selected from SEQ ID N°16, SEQ ID N°17 and SEQ ID N°18, or which differs from a moiety selected from the group of SEQ ID N°16, SEQ ID N°17, and SEQ ID NO: 18 in virtue of 1, 2, 3, 4, or 5 nucleotide modifications.

In other embodiments, the aptamer of the invention is of formula (I):

5′-[NUC1]ₘ-[CENTRAL]-[NUC2]ₙ-3′ (I)

Wherein
n and m are integers independently selected from 0 and 1,
[NUC1] is a polynucleotide comprising from 2 to 40 nucleotides, preferably from 15 to 25 nucleotides. For instance, [NUC1] comprises a polynucleotide of SEQ ID NO:19, or which differs from a polynucleotide of SEQ ID NO:19, in virtue of 1, 2, 3, 4, or 5 nucleotide modifications,
[NUC2] is a polynucleotide comprising from 2 to 40 nucleotides, preferably from 15 to 25 nucleotides. For instance, [NUC2] comprises a polynucleotide of SEQ ID NO:20, or which differs from a polynucleotide of SEQ ID NO:20, in virtue of 1, 2, 3, 4 or 5 nucleotide modifications,
[CENTRAL] is a polynucleotide having at least 70% of sequence identity with a nucleotide sequence selected from the group consisting of SEQ ID NO 1-15 and/or comprising a polynucleotide selected from the group consisting of SEQ ID NO: 16, SEQ ID NO: 17 and SEQ ID NO: 18.

In some embodiments, [CENTRAL] is a polynucleotide of SEQ ID NO: 1-15, or differs from SEQ ID NO: 1-15 in virtue of 1, 2, 3, 4, 5, 6, 7, 8 or 9 nucleotide modifications.

In another embodiment, the aptamer of the invention is of formula (A):

5′-[SEQ ID NO:19]-[X]-[SEQ ID NO:20]-3′ (A)

Wherein:
[SEQ ID NO:19] refers to the polynucleotide of SEQ ID NO:19,
[SEQ ID NO:20] refers to the polynucleotide of SEQ ID NO:20, and
[X] is a polynucleotide selected from the group consisting of SEQ ID NO:1-15.

For instance, the aptamer of the invention can specifically binds to human plasma IgG or recombinant human IgG.

Another object of the invention is an affinity ligand capable of specifically binding IgG which comprises an aptamer moiety as defined above and at least one moiety selected from a mean of detection and a mean of immobilization onto a support.

The invention also relates to a solid affinity support comprising thereon a plurality of affinity ligands or a plurality of aptamers as defined above.

Another object of the invention is a method for preparing a purified IgG composition from a starting IgG-containing composition comprising:
a) contacting said starting composition with an affinity support as defined above, in conditions suitable to form a complex between (i) the aptamers or the affinity ligands immobilized on said support and (ii) IgG
b) releasing IgG from said complex, and
c) recovering a purified IgG composition.

In some embodiment, step a) is performed at a pH lower than 7.0, preferably at a pH from 5.0 to 5.7, and step b) is performed at a pH above 7.0, preferably at pH from 7.2 to 7.6

In some additional or alternate embodiments, step a)-c) are performed by using column or batch chromatography technology.

The invention also relates to the use of an aptamer, an affinity ligand or an affinity support as defined above in the purification of IgG, in the detection of IgG or in blood plasma fractionation process.

In a further aspect, the invention relates to a blood plasma fractionation process comprising:

(a) an affinity chromatography step to recover fibrinogen wherein the affinity ligand is preferably an aptamer which specifically binds to fibrinogen,
(b) an affinity chromatography step to recover immunoglobulins of G isotype (IgG) wherein the affinity ligand is an aptamer which specifically bind to IgG, preferably as defined herein, and
(c) optionally a purification step of albumin,
wherein steps (a), (b) and (c) can be performed in any order.

### BRIEF DESCRIPTION OF THE DRAWINGS

FIG. 1 shows the SELEX protocol used to identify aptamers directed against Fc fragment of human IgG.

FIG. 2 shows the alignments of the central regions (namely SEQ ID NO:1-15) of the 15 aptamers selected by the SELEX process of the invention.

FIG. 3 show the binding properties of some aptamers directed against human IgGs obtained by the method of the invention

FIG. 3A shows the binding curves of human polyclonal IgG (sensorgram) for aptamer A6-2 (namely, SEQ ID NO:1 flanked by primers of SEQ ID NO:19 and SEQ ID NO: 20) and aptamer A.6-8 (SEQ ID NO:2 flanked by primers of SEQ ID NO:19 and SEQ ID NO: 20), immobilized on a sensor chip, obtained by SPR technology. Purified (>95%) human polyclonal IgG (200 nM) was injected at pH 5.50, whereby a complex was formed as evidenced by the increase of the signal. The injection of a buffer solution at pH 5.50 comprising 2M NaCl did not significantly induce the elution of human polyclonal IgG. Human polyclonal IgG was then released from the complex by an elution buffer at pH 7.40. The solid support was then regenerated by injecting a solution of NaOH at 50 mM. X-axis: time in s. Y-axis: SPR response in arbitrary scale

FIG. 3B shows SPR sensograms illustrating the pH dependency of binding of polyclonal IgG to immobilised aptamer of SEQ ID NO:1 flanked by its primer regions of SEQ ID NO:19 and SEQ ID NO: 20 (A6-2). Polyclonal IgG is injected at different pH (in duplicates), after sample injection a running buffer at pH 5.50 is passed over the flow cell in every run. The highest binding level is obtained for pH 5.30. The binding level decreases when pH increases. X-axis: time in s. Y-axis: SPR response in arbitrary scale.

FIG. 4A shows the chromatographic profile for plasma and pre-purified IgG on an affinity support grafted with aptamer of SEQ ID NO:1 flanked by its primers of SEQ ID NO:19 and SEQ ID NO: 20 (A6-2). Y-axis: absorbance at 280 nm. X-axis: in mL

FIG. 4B shows the picture of the electrophoresis gel after coomassie blue staining. From left to right: 1: human plasma, 2: fraction from the plasma which was not retained on the stationary phase grafted with aptamer A6-2, 3: elution fraction containing IgGs obtained from the chromatography of plasma, 4: positive control (plasma IgG) and 5: molecular weight markers.

FIG. 5 shows the binding curves of plasma IgG's sub-classes (sensorgrams) for aptamer of SEQ ID NO:1 flanked by its primers of SEQ ID NO:19 and SEQ ID NO: 20 (A6-2), immobilized on a chip, obtained by SPR technology. Plasma IgG's sub-classes were injected at pH 5.5 (in duplicates), whereby a complex was formed as evidenced by the increase of the signal. The injection of a buffer solution at pH 5.50 comprising 2M NaCl did not significantly induce the elution of human plasma IgG's sub-classes, except for subclass IgG3. The solid support was then regenerated by injection of a solution of NaOH at 50 mM. X-axis: time in s. Y-axis: SPR response in arbitrary scale.

FIG. 6 shows the binding curves of plasma IgG's sub-classes (sensorgrams) for aptamer of SEQ ID NO:7 flanked by its primers of SEQ ID NO:19 and SEQ ID NO: 20 (A6-4), immobilized on a chip, obtained by SPR technology. Plasma IgG's sub-classes were injected at pH 5.5 (in duplicates), whereby a complex was formed as evidenced by the increase of the signal. The injection of a buffer solution at pH 5.50 comprising 2M NaCl did not significantly induce the elution of human plasma IgG's sub-classes. The solid support was then regenerated by injection of a solution of NaOH at 50 mM. X-axis: time in s. Y-axis: SPR response in arbitrary scale.

FIG. 7 shows the binding curves of plasma IgG's sub-classes (sensorgrams) for aptamer of SEQ ID NO:2 flanked by its primers of SEQ ID NO:19 and SEQ ID NO: 20 (A6-8), immobilized on a chip, obtained by SPR technology. Plasma IgG's sub-classes were injected at pH 5.5 (in duplicates), whereby a complex was formed as evidenced by the increase of the signal. The injection of a buffer solution at pH 5.50 comprising 2M NaCl did not significantly induce the elution of human plasma IgG's sub-classes, except for subclass IgG3. The solid support was then regenerated by injection of a solution of NaOH at 50 mM. X-axis: time in s. Y-axis: SPR response in arbitrary scale.

FIG. 8 shows the binding curves of plasma IgG's sub-classes (sensorgrams) for aptamer of SEQ ID NO:11 flanked by its primers of SEQ ID NO:19 and SEQ ID NO: 20 (A6-3), immobilized on a chip, obtained by SPR technology. Plasma IgG's sub-classes were injected at pH 5.5 (in duplicates), whereby a complex was formed as evidenced by the increase of the signal. A considerably faster association rate was observed for sub-classes IgG4 and IgG2 than for IgG1 and IgG3. The injection of a buffer solution at pH 5.50 comprising 2M NaCl did significantly induce the elution of human plasma IgG's sub-classes 1 and 3, and to some extend IgG4, leaving only IgG's sub-classes 2 resistant to 2M NaCl washes. The solid support was then regenerated by injection of a solution of NaOH at 50 mM. X-axis: time in s. Y-axis: SPR response in arbitrary scale.

FIGS. 9A, 9B and 9C show the binding curves of human plasma polyclonal IgG for aptamers of SEQ ID NO: 1, SEQ ID NO:7 and SEQ ID NO:2 flanked by their primers of SEQ ID NO: 19 and SEQ ID NO: 20 (A6-2, A6-4 and A6-8 respectively), immobilized on a sensor chip, obtained by SPR technology. Polyclonal IgGs (200 nM) was injected (in duplicates) using a buffer without Mg2+ (MESBS) or with Mg2+ (MESBS-M5).

FIGS. 10A and 10B show the binding curves of recombinantly produced monoclonal Anti-CD303 IgG (sensorgram) for aptamers of SEQ ID NO:1 and SEQ ID NO:7 flanked by their primers of SEQ ID NO:19 and SEQ ID NO: 20 (A6-2 and A6-4 respectively), immobilized on a sensor chip, obtained by SPR technology. Anti-CD303 IgG (200 nM) was injected (in duplicates) whereby a complex was formed as evidenced by the increase of the signal. The injection of a buffer solution at pH 5.50 comprising 2M NaCl did not significantly induce the elution of Anti-CD303 IgG (ClairYg (200 nM), plasma IgG, was injected as a control). The solid support was then regenerated by injection of a solution of NaOH at 50 mM. X-axis: time in s. Y-axis: SPR response in arbitrary scale.

FIG. 11A shows the chromatographic profile for plasma obtained on an affinity support grafted with aptamer of SEQ ID NO:22 (core sequence of A6-4). Y-axis: absorbance at 280 nm. X-axis: in mL

FIG. 11B shows the distribution of IgG subclasses in the starting composition (pre-purified IgG or plasma) and in the elution fractions obtained by affinity chromatography with the aptamer of SEQ ID NO:22 (from a starting load of 25 g of pre-purified IgG by L of gel, 8 g of pre-purified IgG per L of gel and from plasma, respectively). The elution fractions show a IgG's subclasses distribution close to that of its corresponding starting composition.

FIG. 12 shows the binding curve of purified plasma IgG (sensorgram) for aptamer ATW0018 from Base Pair technologies using the binding buffer recommended by the manufacturer, namely PBS buffer containing 1 mM MgCl₂. No binding was observed. X-axis: time in s. M-axis: SPR response in arbitrary scale. Remarks MESBS buffer refers to 50 mM MES, 150 mM NaCl pH 5.50. MESBS-M5 buffer refers to MESBS, 5 mM MgCl₂ pH 5.50

### DETAILED DESCRIPTION OF THE INVENTION

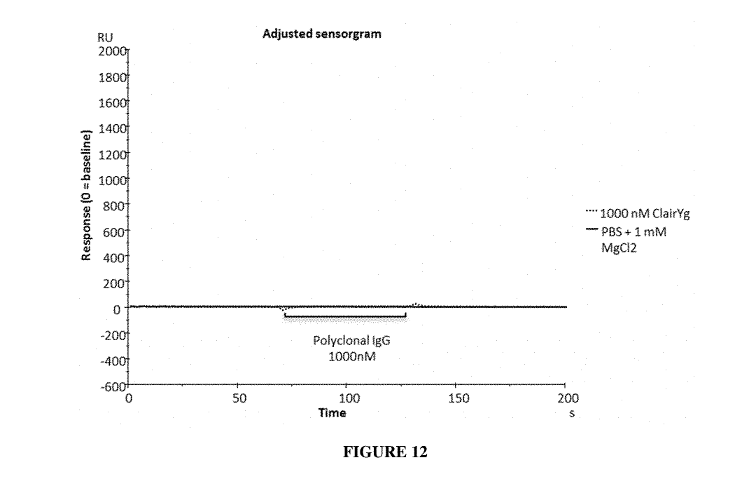

Base Pair Biotechnologies markets IgG Fc C02 aptamers (reference CO2 oligo#369) presented as anti-IgG ligands for research use only. The Applicants investigated the ability of said aptamers to be used as affinity ligands for the purification of IgG. The experiments performed by the Applicant demonstrated that said aptamer did not bind to human polyclonal IgG with the binding buffer recommended by the manufacturer, which precludes its use as affinity ligand in purification process (see FIG. 12).

The Applicant performed his own research and identified a new family of aptamers directed against IgG. This new family of aptamers were identified by an in-house SELEX process conceived by the Applicant. These aptamers were shown to specifically bind both transgenic and plasma human IgG, regardless the glycosylation status of the protein. The aptamers identified by the Applicant display unique properties in terms of binding. In particular, the aptamers of the invention bind to IgG in a pH-dependent manner. Noteworthy they display increased binding affinity for IgG at an acid pH such as a pH of about 5.5 as compared to a pH of 7.4, and even 6.5. Such properties are particularly suitable for use in affinity chromatography because the formation of the complex between the protein to purify, namely IgG, and the aptamer, and the subsequent release of the protein from the complex can be controlled by modifying the pH of the elution buffer. In particular, the release of IgG from the complex can be performed in mild conditions of elution, which are not likely to alter the properties of the protein. Moreover, the aptamers of the invention may be able to bind specifically to several subclasses of human IgG. When used as affinity ligand in chromatography, the aptamers of the invention may enable to retain the distribution of IgG's subclasses in the elution fraction as compared to the starting composition.

The aptamers of the invention can be also used as ligands for diagnostic and detection purposes, even in complex medium such as plasma.

Aptamers of the Invention

Accordingly, the invention relates to an aptamer directed against IgG, i.e. able to specifically bind to IgG. The aptamers of the invention may bind to IgG in a pH-dependent manner. Preferably, the aptamers of the invention do not bind to IgG at a pH higher than 7.0, preferably higher than 6.5, and bind to IgG at an acidic pH below than 6.5, preferably at a pH value selected from 5.0 to 6.0, for instance from 5.2 to 5.8 such as pH 5.5±0.1.

Preferably, the aptamers of the invention are suitable as affinity ligands in the purification of IgG, for instance by chromatography.

Notably, the aptamers of the invention bind to the Fc domain of a IgG.

Thus, in a more general aspect, the aptamers of the invention are suitable as affinity ligands in the purification of a protein comprising a Fc domain from a IgG.

As used herein, an “aptamer” (also called nucleic aptamer) refers to a synthetic single-stranded polynucleotide typically comprising from 20 to 150 nucleotides in length and able to bind with high affinity a target molecule. The aptamers are characterized by three-dimensional conformation(s) which may play a key role in their interactions with their target molecule. Accordingly, the aptamer of the invention is capable of forming a complex with IgG. The interactions between an aptamer and its target molecule may include electrostatic interactions, hydrogen bonds, and aromatic stacking shape complementarity.

“An aptamer specifically binds to its target molecule” means that the aptamer displays a high affinity for the target molecule. The dissociation constant (Kd) of an aptamer for its target molecule is typically from 10⁻⁶ to 10⁻¹² M. The term “specifically binding” is used herein to indicate that the aptamer has the capacity to recognize and interact specifically with its target molecule, while having relatively little detectable reactivity with other molecules which may be present in the sample. Preferably, the aptamer specifically binds to its target molecule if its affinity is significantly higher for the target molecule, as compared to other molecules, including molecules structurally close to the target molecule.

For instance, an aptamer might be able to specifically bind to a human protein while displaying a lower affinity for a homolog of said human protein.

As used herein, “an aptamer display a lower affinity for a given molecule as compared to its target molecule” or “an aptamer is specific to its target molecule as compared to a given molecule” means that the Kd of the aptamer for said given molecule is at least 5-fold, preferably, at least 10, 20, 30, 40, 50, 100, 200, 500, or 1000-fold higher than the Kd of said aptamer for the target molecule.

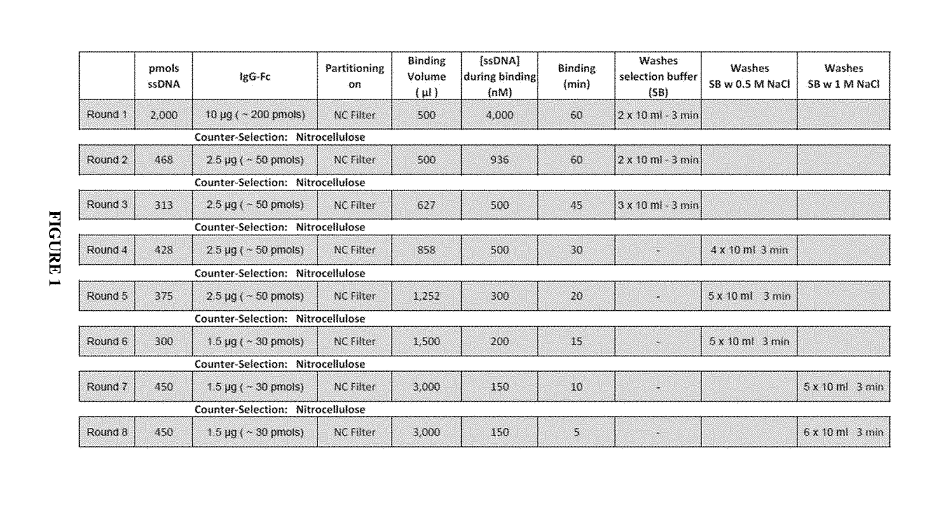

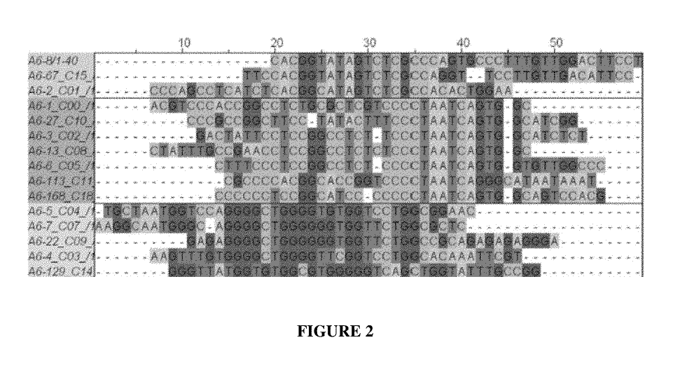

The aptamers may be a deoxyribonucleic acid (DNA) or a ribonucleic acid (RNA). The aptamers can comprise one or several chemically-modified nucleotides. Chemically-modified nucleotides encompass, without being limited to 2′-amino, or 2′ fluoro nucleotides, 2′-ribopurine, phosphoramidite, locked nucleic acid (LNA), boronic acid-modified nucleotides, 5-iodo or 5-bromo-uracil, and 5-modified deoxyuridine such as benzyl-dU, isobutyl-dU, and naphtyl-dU. For 5-modified deoxyuridine, one can refer to Rohloff et al., Molecular Therapy-Nucleic acids, 2014, 3, e201 (see FIG. 1 page 4), the disclosure of which being incorporated herein by reference. In some embodiments, the aptamer of the invention is devoid of any boronic acid-modified nucleotides. In some other embodiments, the aptamer of the invention is devoid of any 5-modified deoxyuridine.

In certain embodiments, the aptamer may comprise a modified nucleotide at its 3′-extremity or/and 5′-extremity only (i.e. the first nucleotide and/or the last nucleotide of the aptamer is/are the sole chemically-modified nucleotide(s)). Preferably, said modified nucleotide may enable the grafting of the aptamer onto a solid support, or the coupling of said aptamer with any moiety of interest (e.g. useful for detection or immobilization).

Once the sequence of the aptamer is identified, the aptamer can be prepared by any routine method known by the skilled artisan, namely by chemical oligonucleotide synthesis, for instance in solid phase.

As used herein, “an aptamer directed to IgG” or “an anti-IgG aptamer” refers to a synthetic single-stranded polynucleotide which specifically binds to at least one IgG, more precisely at least one IgG subclass. Preferably, the anti-IgG aptamer of the invention binds to a IgG on its Fc region.

By “immunoglobulin”, “Ig” or “full-length antibodies” as used herein is meant the structure that constitutes the natural biological form of an antibody, including variable and constant regions. “Full length antibody” covers monoclonal full-length antibodies, wild-type full-length antibodies, chimeric full-length antibodies, humanized full-length antibodies, the list not being limitative. In most mammals, including humans and mice, the structure of full-length antibodies is generally a tetramer. Said tetramer is composed of two identical pairs of polypeptide chains, each pair having one “light” (typically having a molecular weight of about 25 kDa) and one “heavy” chain (typically having a molecular weight of about 50-70 kDa).

In the case of human immunoglobulins, light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. Thus, “isotype” as used herein is meant any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE.

As used herein, the term “IgG” encompasses the four human subclasses of IgG (IgG1, IgG2, IgG3, IgG4) and any protein having the amino acid sequence of a wild-type IgG and variants thereof, regardless the glycosylation state. The term “IgG” encompasses any isoforms or allelic variants of IgG, as well as fragments of IgG such as Fc region, any glycosylated forms, non-glycosylated forms or post-translational modified forms of IgG.

As used herein, a variant of a wild-type IgG refers to a protein having at least 80% of sequence identity, preferably at least 85%, 90%, or 95% of sequence identity with said wild-type IgG and which displays a similar biological activity as compared to said wild-type IgG. The biological activity of a wild-type IgG encompasses complement dependent cytotoxicity (CDC), antibody dependent cytotoxicity (ADCC) or antibody dependent cellular phagocytosis (ADCP) assays. The IgG variant may have an increased or a decreased biological activity, for instance in terms of CDC, or ADCC, or an increased half-life as compared to the corresponding wild-type IgG. In some embodiments, the IgG refers to a protein having the amino acid sequence of a human wild-type IgG, a fully-human IgG or a variant thereof, including chimeric IgG and humanized IgG. Said IgG may be a human plasma IgG, a recombinant or transgenic human IgG as well as a chimeric or a humanized IgG. In some embodiments, the aptamer of the invention is able to bind a human IgG, regardless its glycosylation. For instance an aptamer of the invention may be able to specifically bind to human plasma IgG and a recombinant IgG comprising a Fc domain of a human IgG, for instance a recombinant human IgG, a chimeric IgG or a humanized IgG produced in a recombinant host cell or in a transgenic multicellular organism.

In a more general aspect, an aptamer of the invention can be able to bind to a protein selected from plasma human IgG, fully-human IgG, chimeric IgG, humanized IgG, variants of human IgG, Fc fragment from human IgG and a variant of a Fc from human IgG.

As used herein, “chimeric IgG” and “humanized IgG” refer to IgGs that combine regions from more than one species. “Chimeric IgG” traditionally comprises variable region(s) from a non-human animal, generally the mouse (or rat, in some cases) and the constant region(s) from a human. Humanized IgG are chimeric IgG that contain minimal sequence derived from non-human IgG. Generally, in a humanized IgG, the entire antibody, except the CDRs, is encoded by a polynucleotide of human origin or is identical to a human antibody except within its CDRs. In other words, both chimeric and humanized IgG comprise a Fc domain from a human IgG or a variant thereof.

As used herein, “Fc”, “Fc Fragment” or “Fc region” of IgG refers to the polypeptide comprising the constant region of an antibody excluding the first constant region immunoglobulin domain. Thus Fc refers to the last two constant region immunoglobulin domains of IgG and the flexible hinge N-terminal to these domains.

As used herein, “a protein comprising a Fc domain from a IgG” refers to any artificial, recombinant or naturally-occurring protein or protein construct comprising a Fc domain, or a fragment or a variant thereof, derived from a IgG. Preferably, the Fc domain is a Fc domain derived from a human IgG or a variant thereof. “Proteins comprising a Fc domain from a IgG” encompass immunoglobulins of G isotype, chimeric IgG, humanized IgG, multi-specific antibodies, Fc-fusion proteins and Fc-conjugate proteins. In a preferred embodiment, the Fc domain of said protein is from a human IgG.

The aptamers of the invention may be able to specifically bind to IgG at pH 5.5.

Preferably, the aptamer of the invention displays a constant dissociation (Kd) for a human plasma IgG or for a transgenic human IgG of at most 10⁻⁶ M. Typically, the Kd of the aptamers of the invention for human IgG may be from 1.10⁻¹² M to 1.10⁻⁶ M at a pH of about 5.5. Kd is preferably determined by surface plasmon resonance (SPR) assay in which the aptamer is immobilized on the biosensor chip and Ig is passed over the immobilized aptamers, at a pH of interest, and at a various concentrations, under flow conditions leading to measurement of Kₒₙ and K_{off} and thus Kd. On can refer to the protocol provided in Example 1.

In some embodiments, the aptamer of the invention is specific to a human IgG as compared to a non-human IgG. In some other embodiments, the aptamer of the invention is specific to human IgG as compared to other proteins present in plasma, such as clotting factors, IgA, IgM, IgD and IgE.

In some alternate or additional embodiments, the aptamer of the invention has a higher affinity for IgG at pH 5.5 than at pH 7.4, and in particular a higher affinity for IgG at pH 5.5, as compared to a pH higher than 6.5.

In some alternate or additional embodiments, the aptamers of the invention may have specific affinity to one or several IgGs subclasses, namely IgG1, IgG2, IgG3 and IgG4. For instance, the aptamer of the invention may be able to specifically bind to at least 2 different subclasses, and even to the 4 IgG subclasses. In some other embodiments, the aptamers of the invention may display a higher affinity for one IgG subclass, as compared to other IgG subclasses. For instance, the aptamers may display a higher affinity for IgG2, and eventually IgG4, as compared to other IgG subclasses. In some other embodiment, the aptamers of the invention may specifically bind to IgG1, IgG2, IgG3 and IgG4. This is the case for instance for aptamers A6-2, A6-4 and A6-8 (namely aptamers of formula (A) as described below wherein [X] is SEQ ID NO: 1, SEQ ID NO: 7 or SEQ ID NO: 2, respectively. In some embodiments, when used as affinity ligands in purification, the aptamers of the invention may enable to obtain an elution fraction of purified IgG showing a IgG's subclasses distribution similar to that of the starting composition.

In some other embodiments, the binding of the aptamer for IgG may be increased in the presence of Mg²⁺, for instance at a concentration in the mM, such as 1 to 10 mM, as compared the same medium devoid of Mg²⁺. In some other embodiments, the binding of the aptamer for IgG may be decreased in the presence of Mg²⁺, for instance at a concentration in the mM, such as 1 to 10 mM, as compared the same medium devoid of Mg²⁺. In some other embodiments, the binding of the aptamer to IgG is not significantly modified in the presence or the absence of Mg²⁺. In some embodiments, the binding of an aptamer of the invention to a IgG does not require the presence of Ca²⁺. In other words, the aptamer of the invention may be not dependent of Ca²⁺. For instance, when the aptamer of the invention is used to purify IgG from plasma or plasma fractions by chromatography, the binding buffer and/or the elution buffer may be devoid of Ca²⁺.

As mentioned above, the Applicant identified aptamers which specifically bind to a IgG, in particular to the Fc region of a IgG, by performing an in-house SELEX method on a ssDNA library wherein the ssDNA consisted of a 40-base random regions flanked by two constant 18-base primer regions (namely SEQ ID NO:19 and 20). More precisely, the Applicant identified 15 aptamers of interest having the following formula (A):

5′-[SEQ ID NO:19]-[X]-[SEQ ID NO:20]-3′ (A)

Wherein:
[SEQ ID NO:19] refers to the polynucleotide of SEQ ID NO:19,
[SEQ ID NO:20] refers to the polynucleotide of SEQ ID NO:20, and
[X] is a polynucleotide selected from the group consisting of SEQ ID NO:1-15.

By performing sequence alignments on these 15 aptamers, the Applicant identified the consensus sequence moieties of SEQ ID NO: 16, SEQ ID NO:17 and SEQ ID NO:18.

Thus, in a certain aspect, the invention relates to an aptamer capable of specifically binding to IgG and comprising a moiety selected from the group consisting of SEQ ID N°16, SEQ ID N°17 and SEQ ID N°18, or which differs from a moiety selected from the group of SEQ ID N°16, SEQ ID N°17, and SEQ ID NO: 18 in virtue of 1, 2, 3, 4, or 5 nucleotide modifications. In some embodiments, the aptamer of the invention comprises a polynucleotide:
having at least 70%, such as at least 75%, 80%, 85%, 90%, or 95% of identity with a sequence selected from the group of SEQ ID NO:1-15 and
comprising a moiety selected from SEQ ID N°16, SEQ ID N°17 and SEQ ID N°18, or which differs from a moiety selected from the group of SEQ ID N°16, SEQ ID N°17, and SEQ ID NO: 18 in virtue of 1, 2, 3, 4, or 5 nucleotide modifications.

As used herein, a “nucleotide modification” refers to the deletion of a nucleotide, the insertion of a nucleotide, or the substitution of a nucleotide by another nucleotide as compared to the reference sequence.

The Applicant also determined the core sequence for aptamers A.6-2, A6-4 and A6-8. A.6-2, A6-4 and A6-8 refer to aptamers of formula (A) wherein [X] is SEQ ID NO: 1, SEQ ID NO: 7 and SEQ ID NO:2, respectively. The core sequence for aptamer A.6-2 is the polynucleotide of SEQ ID NO: 21. The core sequence for aptamer A.6-4 is the polynucleotide of SEQ ID NO: 22. The core sequence for aptamer A.6-8 is SEQ ID NO: 23. As used herein, a “core sequence” of a given aptamer typically comprises, or refers to, the minimal sequence issued from said aptamer able to bind IgG.

In another aspect, the invention relates to an aptamer capable of specifically binding to IgG and comprising a polynucleotide having at least 70% of sequence identity with a nucleotide sequence selected from the group consisting of SEQ ID N°21, SEQ ID N°22 and SEQ ID N°23. In some embodiments, said polynucleotide having at least 70% of sequence identity with a nucleotide sequence selected from the group consisting of SEQ ID N°21, SEQ ID N°22 and SEQ ID N°23, further comprises a moiety selected from SEQ ID N°16, SEQ ID N°17 and SEQ ID N°18, or which differs from a moiety selected from the group of SEQ ID N°16, SEQ ID N°17, and SEQ ID NO: 18 in virtue of 1, 2, or 3 nucleotide modifications.

As used herein, a sequence identity of at least 70% encompasses a sequence identity of at least 75%, 80%, 85%, 86%, 87%, 88%, 89%, 90%, 91%, 92%, 93%, 94%, 95%, 96%, 97%, or 98%. The “percentage identity” between two nucleotide sequences (A) and (B) may be determined by comparing the two sequences aligned in an optimal manner, through a window of comparison. Said alignment of sequences can be carried out by well-known methods, for instance, using the algorithm for global alignment of Needleman-Wunsch. Once alignment is obtained, the percentage of identity can be obtained by dividing the full number of identical amino acid residues aligned by the full number of residues contained in the longest sequence between the sequence (A) and (B). Sequence identity is typically determined using sequence analysis software. For comparing two nucleic acid sequences, one can use, for example, the tool “Emboss needle” for pairwise sequence alignment of providing by EMBL-EBI and available on http://www.ebi.ac.uk/Tools/psa/emboss_needle/nucleotide.html using default settings: (I) Matrix: DNAfull, (ii) Gap open: 10, (iii) gap extend: 0.5, (iv) output format: pair, (v) end gap penalty: false, (vi) end gap open: 10, (vii) end gap extend: 0.5.

The aptamer of the invention typically comprises from 20 to 150 nucleotides in length, preferably from 30 to 100 nucleotides in length, for instance from 25 to 90 nucleotides in length. Accordingly, the aptamer of the invention may have 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 in length.

Typically the aptamer of the invention may have from 30 to 80 nucleotides in length.

The aptamers of the invention may also comprise primers at its 3′- and 5′-terminus useful for its amplification by PCR. In some embodiments, these primer sequences can be included or partially included in the core sequence and thus participate in binding interactions with IgG. In some other embodiments, these primer sequences are outside the core sequence and may not play any role in the interaction of the aptamer with IgG. In some further embodiments, the aptamer is devoid of primer sequences.

In some alternate or additional embodiments, the aptamer of the invention may comprise a polynucleotide of 2 to 40 nucleotides in length linked to the 5′-end and/or the 3′-end of the core sequence.

In some embodiments, the aptamer of the invention is of formula (I)

5′-[NUC1]ₘ-[CENTRAL]-[NUC2]ₙ-3′

Wherein

n and m are integers independently selected from 0 and 1,
[NUC1] is a polynucleotide comprising from 2 to 40 nucleotides, preferably from 15 to 25 nucleotides
[NUC2] is a polynucleotide comprising from 2 to 40 nucleotides, preferably from 15 to 25 nucleotides and
[CENTRAL] is a polynucleotide having at least 70% of sequence identity with a nucleotide sequence selected from the group consisting of SEQ ID NO 1-15 and/or comprising a polynucleotide selected from the group consisting of SEQ ID NO: 16, SEQ ID NO: 17 and SEQ ID NO: 18.

When n=m=0, [NUC1] and [NUC2] are absent and the aptamer consists of the central sequence [CENTRAL].

When n=0 and m=1, [NUC2] is absent and [NUC1] is present, the aptamer is thus of formula (Ia):

5′-[NUC1]-[CENTRAL]-3′.

When n=1 and m=0, [NUC1] is absent and [NUC2] is present, the aptamer is thus of formula (Ib):

5′-[CENTRAL]-[NUC2]-3′.

In some embodiments, [NUC1] comprises, or consists of, a polynucleotide of SEQ ID N°19 or a polynucleotide which differs from SEQ ID N°19 in virtue of 1, 2, 3, or 4 nucleotide modifications.

In some other or additional embodiments, [NUC2] comprises, or consists of, a polynucleotide of SEQ ID N°20 or a polynucleotide which differs from SEQ ID N°20 in virtue of 1, 2, 3, or 4 nucleotide modifications.

In a further aspect, the invention relates an aptamer directed against IgG and which has at least 70%, such as at least 75%, 80%, 85%, 90%, 95%, 96%, 97% or 98% of sequence identity with a polynucleotide of formula (A) as described above, preferably wherein [X] is selected from SEQ ID NO: 1, SEQ ID NO: 2, SEQ ID NO: 7 and SEQ ID NO: 11. In some embodiments, said aptamer may further comprise a moiety selected from SEQ ID NO: 16, SEQ ID NO: 17 and SEQ ID NO: 18.

In another aspect, the invention relates to an aptamer capable of specifically binding IgG and which comprises the nucleotide moiety of formula (IV):

5′-[SEQ ID NO:19]-[X1]-[SEQ ID NO:16-18]-[X2]-[SEQ ID NO:20]-3′

Wherein:
[X1] and [X2] independently denote a nucleotide or an oligonucleotide of 0 to 25 nucleotides in length

[SEQ ID NO: 19] is an oligonucleotide of SEQ ID
NO: 19
(namely GGGTCAATGCCAGGTCTC)
[SEQ ID NO: 20] is an oligonucleotide of SEQ ID
NO: 20
(namely ATCGGCTCGCAAGCAGTC)
[SEQ ID NO: 16-18] is oligonucleotide selected
from SEQ ID NO: 16, SEQ ID NO: 17 and SEQ ID
NO: 18 respectively
(namely CACGGTATAGTCTCGCCA;
AGGGGCTGGGGTGTGGTTCTGGC; CCCCTAATCAGTGGC).

The Applicant performed an analysis of the sequences. This analysis led to the identification of three subgroups of aptamers, each subgroup being characterized by specific structural and functional properties.

First Subgroup of Aptamers According to the Invention

The first subgroup of aptamers encompasses aptamers directed against IgG which comprises the consensus sequence moiety of SEQ ID N°16. This first subgroup encompass aptamers of formula (A) wherein [X] is selected from SEQ ID NO: 1, SEQ ID NO: 2 and SEQ ID NO:3 and aptamers consisting of the core sequence of SEQ ID NO: 21 and SEQ ID NO: 23. Accordingly, the invention also relates to an aptamer which selectively binds to IgG and which comprises the consensus moiety of SEQ ID NO:16 or a moiety which differs from SEQ ID NO:16 in virtue of 1, 2, 3, 4 or 5, preferably 1, 2 or 3 nucleotide modifications.

In some embodiment, the aptamer comprises a polynucleotide
having at least 70%, 75%, 80%, 85%, 90%, 92%, 94%, 95%, 96%, 97% or 98% of sequence identity with SEQ ID N°1, SEQ ID NO: 2, SEQ ID N:3, SEQ ID NO: 21 and SEQ ID NO: 23, and
comprising the consensus moiety of SEQ ID NO:16 or a moiety which differs from SEQ ID NO:16 in virtue of 1, 2, 3, 4 or 5, preferably 1, 2, 3 nucleotide modifications.

Preferably, said aptamer has from 25 to 110 nucleotides in length, in particular from 35 to 80 nucleotides in length, such as 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 in length.

In some embodiments, the aptamer of the invention is of formula (I):

5′-[NUC1]ₘ-[CENTRAL]-[NUC2]ₙ-3′ (I) wherein:
[CENTRAL] is a polynucleotide having at least 80%, preferably at least 85%, more preferably at least 90%, 93%, 95%, 96%, 97%, 98%, 99% or 100% of sequence identity with SEQ ID N°1-3 and/or comprising the consensus sequence moiety of SEQ ID NO: 16,
n and m are integers independently selected from 0 and 1,
[NUC1] is a polynucleotide comprising from 2 to 40 nucleotides, preferably from 15 to 25 nucleotides, and
[NUC2] is a polynucleotide comprising from 2 to 40 nucleotides, preferably from 15 to 25 nucleotides.

In some embodiments, n and m are 0, which means that [NUC1] and [NUC2] are absent. In such a case, the aptamer consists of a polynucleotide having at least 80% of sequence identity with SEQ ID NO: 1-3 and/or comprising the consensus sequence moieties of SEQ ID NO:16. When n=0 and m=1, [NUC2] is absent and [NUC1] is present, the aptamer is thus of formula (Ia):

5′-[NUC1]-[CENTRAL]-3′ (Ia).

When m is 0 and n is 1, [NUC1] is absent and [NUC2] is present. Accordingly, the aptamer of the invention is of the following formula (Ib):

5′-[CENTRAL]-[NUC2]ₙ-3′ (Ib)

In some embodiments, [NUC1] may comprise, or consist of, a polynucleotide of SEQ ID NO: 19 or a polynucleotide which differs from SEQ ID NO: 19 in virtue of 1, 2, 3, or 4 nucleotide modifications.

In some other or additional embodiment, [NUC2] may comprise, or consist of, a polynucleotide of SEQ ID NO: 20 or a polynucleotide which differs from SEQ ID NO: 20 in virtue of 1, 2, 3, or 4 nucleotide modifications.

In some other or additional embodiments of the aptamer of formula (I) as described above, [CENTRAL] is a polynucleotide of SEQ ID NO:1-3, or has a nucleotide sequence which differs from SEQ ID NO: 1-3 in virtue of 1, 2, 3, 4, 5, 6, 7, 8 or 9 nucleotide modifications. As mentioned above, the nucleotide modifications(s) can be of any type. A nucleotide modification may be a deletion of one nucleotide, the insertion of one nucleotide or the substitution/replacement of one nucleotide by another nucleotide.

In some embodiments, the aptamer of the invention may be an aptamer of formula (I) wherein [CENTRAL] is a polynucleotide which comprises or consists of SEQ ID NO: 16, or comprises or consists of a nucleotide sequence which differs from SEQ ID NO: 16 in virtue of 1, 2, 3, 4, or 5 nucleotide modifications, preferably in virtue of 1 or 2 nucleotide substitutions(s), said nucleotide modification(s) being at nucleotide positions selected from 6 and 18, the numbering referring to nucleotide numbering in SEQ ID NO: 16.

In another aspect, the invention relates to an aptamer capable of specifically binding to IgG and which comprises the nucleotide moiety of formula (IVa)

5′-[SEQ ID NO:19]-[X1]-[SEQ ID NO:16]-[X2]-[SEQ ID NO:20]-3′

wherein [X1] and [X2] independently denote a nucleotide or an oligonucleotide of 0 to 25 nucleotides in length.

The aptamers belonging to said first subgroup may be able to bind to IgG at an acidic pH, preferably at a pH of around 5.5. In some embodiments, said aptamers display an increased affinity for IgG at pH 5.5 as compared to a pH such as pH 7.0 or 6.5.

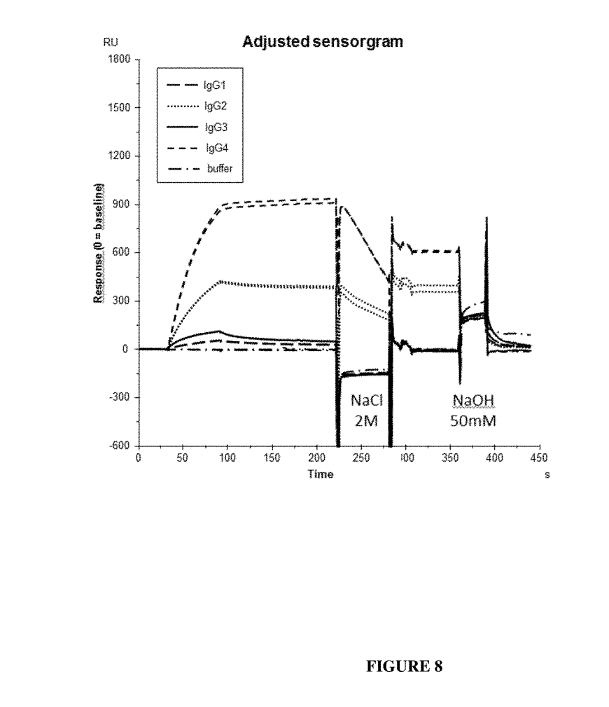

Certain aptamers of said subgroup may be able to bind to IgG in the presence of Mg²⁺. In some embodiments, said aptamers may display a binding affinity for IgG which depends on the pH and/or the presence of Mg²⁺ in the medium. For instance, the binding affinity of the aptamer for the IgG may be increased in the presence of Mg²⁺ at a concentration in the mM range, for instance from 1 to 10 mM, as compared to the same medium devoid of Mg²⁺. This is the case, for example, of aptamer A6-8 (aptamer of formula (A) wherein [X] is SEQ ID NO: 2). Certain aptamers of this subgroup may show a binding affinity for IgG which is not significantly modified by Mg2+. This is the case, for instance, of aptamer A6-2 (aptamer of formula (A) wherein [X] is SEQ ID NO: 2) (see FIG. 9). The aptamers of this subgroup may be independent of Ca²⁺, i.e. they may not require the presence of Ca²⁺ to bind IgG.

As a further example, the aptamer of the invention may display a higher affinity for IgG at a pH of about 5.5 as compared to a higher pH such as pH 7.0.

Such properties are for instance illustrated herein for aptamers A6-2 and A6-8 in the below section entitled “Examples”.

Second Subgroup of Aptamers According to the Invention

The second subgroup of aptamers encompasses aptamers directed against IgG which comprises the consensus sequence moieties of SEQ ID N°17. This second subgroup encompasses aptamers of formula (A) wherein [X] is of SEQ ID NO: 4-8 and the aptamer consisting of the core sequence of SEQ ID NO: 22.

Accordingly, the invention also relates to an aptamer which selectively binds to IgG and which comprises the consensus moiety of SEQ ID NO:17 or a moiety which differs from SEQ ID NO:17 in virtue of 1, 2, 3, 4 or 5, preferably 1, 2 or 3 nucleotide modifications.

In some embodiment, the aptamer comprises a polynucleotide
having at least 70%, 75%, 80%, 85%, 90%, 92%, 94%, 95%, 96%, 97% or 98% of sequence identity with SEQ ID N°4, SEQ ID NO: 5, SEQ ID NO: 6, SEQ ID NO: 7, SEQ ID NO: 8 and SEQ ID NO: 22, and
comprises the consensus moiety of SEQ ID NO:17 or a moiety which differs from SEQ ID NO:17 in virtue of 1, 2, 3, 4 or 5, preferably 1, 2 or 3 nucleotide modifications.

Preferably, said aptamer has from 25 to 110 nucleotides in length, in particular from 35 to 80 nucleotides in length, such as 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 in length. In some alternate or additional embodiments, the aptamer of the invention may comprise a polynucleotide moiety of 2 to 40 nucleotides in length linked to the 5′-end and/or the 3′-end of said polynucleotide.

In some embodiments, the aptamer of the invention is of formula (I) wherein:

5′-[NUC1]ₘ-[CENTRAL]-[NUC2]ₙ-3′ (II)

Wherein
[CENTRAL] is a polynucleotide having at least 80%, preferably at least 85%, more preferably at least 90%, 93%, 95%, 96%, 97%, 98%, 99% or 100% of sequence identity with SEQ ID NO: 4-8 and/or comprising the consensus sequence moiety of SEQ ID NO:17
n and m are integers independently selected from 0 and 1,
[NUC1] is a polynucleotide comprising from 2 to 40 nucleotides, preferably from 15 to 25 nucleotides, and
[NUC2] is a polynucleotide comprising from 2 to 40 nucleotides, preferably from 15 to 25 nucleotides

In some embodiments, n and m are 0, which means that [NUC1] and [NUC2] are absent. In such a case, the aptamer consists of a polynucleotide having at least 80% of sequence identity with SEQ ID NO: 4-8 and/or comprising the consensus sequence moieties of SEQ ID NO:17, When n=0 and m=1, [NUC2] is absent and [NUC1] is present, the aptamer is thus of formula (IIa):

5′-[NUC1]-[CENTRAL]-3′.

When m is 0 and n is 1, [NUC1] is absent and [NUC2] is present. Accordingly, the aptamer of the invention is of the following formula (IIb):

5′-[CENTRAL]-[NUC2]ₙ-3′ (IIb)

In some embodiments, [NUC1] may comprise, or consist of, a polynucleotide of SEQ ID NO: 19 or a polynucleotide which differs from SEQ ID N°19 in virtue of 1, 2, 3, or 4 nucleotide modifications.

In some other or additional embodiments, [NUC2] may comprise, or consist of, a polynucleotide of SEQ ID NO: 20 or a polynucleotide which differs from SEQ ID N°20 in virtue of 1, 2, 3, or 4 nucleotide modifications.

In some other or additional embodiments of the aptamer of formula (II) as described above, [CENTRAL] is a polynucleotide of SEQ ID NO: 4-8, or has a nucleotide sequence which differs from SEQ ID NO: 4-8 in virtue of 1, 2, 3, 4, 5, 6, 7, 8 or 9 nucleotide modifications. As mentioned above, the nucleotide modifications(s) can be of any type. A nucleotide modification may be a deletion of one nucleotide, the insertion of one nucleotide or the substitution/replacement of one nucleotide by another nucleotide.

In some embodiments, the aptamer of the invention may be an aptamer of formula (II) wherein [CENTRAL] is a polynucleotide which comprises or consists of SEQ ID NO: 17, or comprises or consists of a nucleotide sequence which differs from SEQ ID NO: 17 in virtue of 1, 2, 3, 4 or 5 nucleotide modification(s), preferably in virtue of 1, 2 or 3 nucleotide substitutions(s), said nucleotide modification(s) being at nucleotide position(s) selected from the group consisting of 12, 14 and 18 the numbering referring to nucleotide numbering in SEQ ID NO: 17.

In another aspect, the invention relates to an aptamer capable of specifically binding to IgG and which comprises the nucleotide moiety of formula (IVb):

5′-[SEQ ID NO:19]-[X1]-[SEQ ID NO:17]-[X2]-[SEQ ID NO:20]-3′

wherein [X1] and [X2] independently denote a nucleotide or an oligonucleotide of 0 to 25 nucleotides in length.

The aptamers belonging to said second subgroup may be able to bind to IgG at an acidic pH, preferably at a pH of around 5.5. In some embodiments, said aptamers display an increased affinity for IgG at pH 5.5 as compared to a higher pH such as pH 7.0 or 6.5.

Said subgroup of aptamers may be also able to bind to IgG in the presence of Mg²⁺. In some embodiments, said aptamers may display a binding affinity for IgG which depends on the pH and/or the presence of Mg²⁺ in the medium. For instance, the binding affinity of the aptamer for the IgG may be increased in the presence of Mg²⁺ at a concentration in the mM range, for instance from 1 to 10 mM, as compared to the same medium devoid of Mg²⁺.

This is the case, for instance, of aptamer A.6-4 of formula (A) wherein [X] is SEQ ID NO: 7 (see FIG. 9). As a further example, the aptamer of the invention may display a higher affinity for IgG at a pH of about 5.5 as compared to pH 7.0. The aptamers of this subgroup may be also independent of Ca²⁺, i.e. they may not require the presence of Ca²⁺ to bind IgG.

Third Subgroup of Aptamers According to the Invention

The third subgroup of aptamers encompasses aptamers directed against IgG which comprises the consensus sequence moieties of SEQ ID NO: 18. This third subgroup encompass aptamers of formula (A) wherein [X] is selected from SEQ ID NO: 9-15.

Accordingly, the invention also relates to an aptamer which selectively binds to IgG and which comprises the consensus moiety of SEQ ID NO:18 or a moiety which differs from SEQ ID NO:18 in virtue of 1, 2, 3, 4 or 5, preferably 1, 2 or 3 nucleotide modifications.

In some embodiment, the aptamer comprises a polynucleotide
having at least 70%, 75%, 80%, 85%, 90%, 92%, 94%, 95%, 96%, 97% or 98% of sequence identity with SEQ ID NO: 9, SEQ ID NO: 10, SEQ ID NO: 11, SEQ ID NO: 12, SEQ ID NO: 13, SEQ ID NO: 14 and SEQ ID NO: 15 and
comprising the consensus moiety of SEQ ID NO:18 or a moiety which differs from SEQ ID NO:18 in virtue of 1, 2, 3, 4 or 5, preferably 1, 2 or 3 nucleotide modifications.

Preferably, said aptamer has from 25 to 110 nucleotides in length, in particular from 35 to 80 nucleotides in length, such as 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 in length. In some alternate or additional embodiments, the aptamer of the invention may comprise a polynucleotide moiety of 2 to 40 nucleotides in length linked to the 5′-end and/or the 3′-end of said polynucleotide.

In some embodiments, the aptamer of the invention is of formula (III):

5′-[NUC1]ₘ-[CENTRAL]-[NUC2]ₙ-3′ (III)

Wherein:
[CENTRAL] is a polynucleotide having at least 80%, preferably at least 85%, more preferably at least 90%, 93%, 95%, 96%, 97%, 98%, 99% or 100% of sequence identity with SEQ ID N°9-15 and/or comprising the consensus sequence moiety of SEQ ID NO: 18
n and m are integers independently selected from 0 and 1,
[NUC1] is a polynucleotide comprising from 2 to 40 nucleotides, preferably from 15 to 25 nucleotides, and
[NUC2] is a polynucleotide comprising from 2 to 40 nucleotides, preferably from 15 to 25 nucleotides

In some embodiments, n and m are 0, which means that [NUC1] and [NUC2] are absent. In such a case, the aptamer consists of a polynucleotide having at least 80% of sequence identity with SEQ ID NO: 9-15 and/or comprising the consensus sequence moieties of SEQ ID NO: 18. When, m is 0 and n is 1. Accordingly, the aptamer of the invention is of the following formula (III):

5′-[CENTRAL]-[NUC2]ₙ-3′ (II)

When n=0 and m=1, [NUC2] is absent and [NUC1] is present, the aptamer is thus of formula (IIIa):

5′-[NUC1]-[CORE]-3′.

When m is 0 and n is 1, [NUC1] is absent and [NUC2] is present. Accordingly, the aptamer of the invention is of the following formula (IIb):

5′-[CENTRAL]-[NUC2]ₙ-3′ (IIIb)

In some embodiments, [NUC1] may comprise, or consist of, a polynucleotide of SEQ ID NO: 19 or a polynucleotide which differs from SEQ ID N°19 in virtue of 1, 2, 3, or 4 nucleotide modifications.

In some other or additional embodiments, [NUC2] may comprise, or consist of, a polynucleotide of SEQ ID NO: 20 or a polynucleotide which differs from SEQ ID NO: 20 in virtue of 1, 2, 3, or 4 nucleotide modifications.

In some other or additional embodiments of the aptamer of formula (III) as described above, [CENTRAL] is a polynucleotide of SEQ ID NO: 9-15, or has a nucleotide sequence which differs from SEQ ID NO: 9-15 in virtue of 1, 2, 3, 4, 5, 6, 7, 8 or 9 nucleotide modifications. As mentioned above, the nucleotide modifications(s) can be of any type. A nucleotide modification may be a deletion of one nucleotide, the insertion of one nucleotide or the substitution/replacement of one nucleotide by another nucleotide.

In some embodiments the aptamer of the invention may be an aptamer of formula (III) wherein [CENTRAL] is a polynucleotide which comprises or consists of SEQ ID NO: 18, or comprises or consists of a nucleotide sequence which differs from SEQ ID NO: 18 in virtue of 1, 2, 3, 4, or 5 nucleotide modification(s), preferably in virtue of 1 or 2 nucleotide substitutions(s) or deletions, said nucleotide modification(s) being at nucleotide position(s) selected from the group consisting of 1 and 16 the numbering referring to nucleotide numbering in SEQ ID NO: 18.

In another aspect, the invention relates to an aptamer capable of specifically binding IgG and which comprises the nucleotide moiety of formula (IVc)

5′-[SEQ ID NO:19]-[X1]-[SEQ ID NO:18]-[X2]-[SEQ ID NO:20]-3′

wherein [X1] and [X2] independently denote a nucleotide or an oligonucleotide of 0 to 25 nucleotides in length.

The aptamers belonging to said third subgroup may be able to bind to IgG at an acidic pH, preferably at a pH of around 5.5. In some embodiments, said aptamers display an increased affinity for IgG at pH 5.5 as compared to a higher pH such as pH 7.0 or pH 6.5.

Said subgroup of aptamers may be also able to bind to IgG in the presence of Mg²⁺. In some embodiments, said aptamers may display a binding affinity for IgG which depends on the pH and/or the presence of Mg²⁺ in the medium. As a further example, the aptamer of the invention may display a higher affinity for IgG at a pH of about 5.5 as compared to pH 7.0. The aptamers of this subgroup may be also independent of Ca²⁺, i.e. they may not require the presence of Ca²⁺ to bind IgG

Affinity Ligands and Affinity Supports of the Invention

The invention also relates to affinity ligands comprising an aptamer directed against IgG. Said affinity ligands may be immobilized onto a solid support for the detection, the quantification, or the purification of IgG. Alternatively or additionally, the affinity ligands may comprise a mean of detection. A mean of detection may be any compound generating a signal quantifiable, preferably by instrumented reading. Suitable detectable labels may be selected, for example, from the group consisting of colloidal metals such as gold or silver; non-metallic colloids such as colloidal selenium, tellurium or sulphur particles; fluorescent, luminescent and chemiluminescent dyes, fluorescent proteins such as GFP, magnetic particles, radioactive elements, and enzymes such as horseradish peroxidase.

Typically, the affinity ligand of the invention comprises (i) an aptamer moiety, i.e. an aptamer directed against IgG as defined above linked to at least one (ii) non-aptamer entity useful for immobilization on an appropriate substrate. Preferably, the non-entity aptamer is linked to the 5′- or the 3′-end of the aptamer.

In certain embodiment, the affinity ligand may comprise a mean of immobilization linked to the aptamer moiety directly or by a spacer group. Accordingly, the affinity ligand may comprise, or consist of, a compound of formula (IV):

[IMM]-([SPACER])ₚ-[APTAMER] wherein
[APTAMER] denotes an aptamer as defined above,
[SPACER] is a spacer group,
[IMM] is a moiety for the immobilization of the aptamer onto a support and
p is 0 or 1.
   p is 0 means that the spacer is absent and that [IMM] is directly linked to [APTAMER], preferably at the 3′ or the 5′-end of aptamer.
   p is 1 means that the spacer is present and links to [IMM] and [APTAMER].

The spacer group is typically selected to decrease the steric hindrance of the aptamer moiety and improve its accessibility while preserving the aptamer capability of specifically binding to IgG. The spacer group may be of any type. The spacer may be a non-specific single-stranded nucleotide, i.e. which is not able to bind to a protein, including IgG. Typically the spacer may comprise from 2 to 20 nucleotides in length. Examples of appropriate nucleic spacers are polyA and polyT. In some other embodiments, the spacer may be a non-nucleic chemical entity. For instance, the spacer may be selected from the group consisting of a peptide, a polypeptide, an oligo- or polysaccharide, a hydrocarbon chain optionally interrupted by one or several heteroatoms and optionally substituted by one or several substituents such as hydroxyl, halogens, or C₁-C₃ alkyl; polymers including homopolymers, copolymers and block polymers, and combinations thereof. For instance the spacer may be selected from the group consisting of polyethers such as polyethylene glycol (PEG) or polypropylene glycol, polyvinylic alcool, polyacrylate, polymethacrylate, polysilicone, and combination thereof. For instance, the spacer may comprise several hydrocarbon chains, oligomers or polymers linked by any appropriate group, such as a heteroatom, preferably —O— or —S—, —NHC(O)—, —OC(O)—, —NH—, —NH—CO—NH—, —O—CO—NH—, phosphodiester or phosphorothioate. Such spacer chains may comprise from 2 to 200 carbon atoms, such as from 5 to 50 carbon atoms. Preferably, the spacer is selected from non-specific oligonucleotides, hydrocarbon chains, polyethers, in particular polyethylene glycol and combinations thereof.

For instance, the spacer comprises at least one polyethylene glycol moiety comprising from 2 to 20 monomers. For instance, the spacer may comprise from 1 to 4 triethyleneglycol blocks linked together by appropriate linkers. For example, the spacer may be a C12 hydrophilic triethylene glycol ethylamine derivative. Alternatively, the spacer may be a C₂-C₂₀ hydrocarbon chain, in particular a C₂-C₂₀ alkyl chain such as a C₁₂ methylene chain.

The spacer is preferably linked to the 3′-end or the 5-end of the aptamer moiety, preferably linked to the 5′-end of the aptamer moiety.

[IMM] refers to any suitable moiety enabling to immobilize the affinity ligand onto a substrate, preferably a solid support. [IMM] depends on the type of interactions sought to immobilize the affinity ligand on the substrate.

For instance, the affinity ligand may be immobilized thanks to specific non-covalent interactions including hydrogen bonds electrostatic forces or Van der Waals forces. For example, the immobilization of the affinity ligand onto the support may rely ligand/anti-ligand couples (e.g. antibody/antigen such as biotin/anti-biotin antibody and digoxygenine/anti-digoxigenin antibody, or ligand/receptor) and protein binding tags. A multitude of protein tags are well-known by the skilled person and include, for example, biotin (for binding to streptavidin or avidin derivatives), glutathione (for binding to proteins or other substances linked to glutathione-S-transferase), maltose (for binding to proteins or other substances linked to maltose binding protein), lectins (for binding to sugar moieties), c-myc tag, hemaglutinin antigen (HA) tag, thioredoxin tag, FLAG tag, polyArg tag, polyHis tag, Strep-tag, chitin-binding domain, cellulose-binding domain, and the like. In some embodiments, [IMM] denotes biotin. Accordingly, the affinity ligand of the invention is suitable to be immobilized on supports grafted with avidin or streptavidin.

Alternatively, the affinity ligand may be suitable for covalent grafting on a solid support. [IMM] may thus refer to a chemical entity comprising a reactive chemical group. The chemical entity has typically a molecular weight below than 1000 g·mol−1, preferably less than 800 g·mol⁻¹ such as less than 700, 600, 500 or 400 g·mol⁻¹. The reactive groups can be of any type and encompasses primary amine, maleimide group, sulfhydryl group and the likes.

For instance, the chemical entity may derive from SIAB compound, SMCC compound or derivatives thereof. The use of sulfo-SIAB to immobilize oligonucleotides is for instance described in Allerson et al., RNA, 2003, 9:364-374

In some embodiments, [IMM] comprises a primary amino group. For instance, [IMM] may be —NH₂ or a C₁₋₃₀ aminoalkyl preferably a C₁-C₆ aminoalkyl. An affinity ligand wherein [IMM] comprises a primary suitable group is suitable for immobilization on support having thereon activated carboxylic acid groups. Activated carboxylic acid groups encompass, without being limited to, acid chloride, mixed anhydride and ester groups. A preferred activated carboxylic acid group is N-hydroxysuccinimide ester.

As mentioned above, [IMM]-([SPACER])p is preferably links to the 3′-end or the 5′-end of the aptamer. The terminus of the aptamer moiety which is not linked to [IMM]-([SPACER])p may comprise a chemically modified nucleotide such as 2′-o-methyl or 2′ fluoropyrimidine, 2′-ribopurine, phosphoramidite, an inverted nucleotide or a chemical group such as PEG or cholesterol. Such modifications may prevent the degradation, in particular the enzymatic degradation of the ligands. In other embodiments, said free terminus of the aptamer (i.e. which is not bound to [IMM] or to [SPACER]) may be linked to a mean of detection as described above.

A further object of the invention is an affinity support capable of selectively binding IgG, which comprises thereon a plurality of affinity ligands as defined above.

The affinity ligands can be immobilized onto the solid support by non-covalent interactions or by a covalent bond(s).

In some embodiments, the affinity ligands are covalently grafted on said support. Typically, the grafting is performed by reacting the chemical reactive group present in the moiety [IMM] of the ligand with a chemical reactive group present on the surface of the solid support.

Preferably, the chemical reactive group of the ligand is a primary amine group and that present on the solid support is an activated carboxylic acid group such as a NHS-activated carboxylic group (namely N-hydroxysuccimidyle ester). In this case, the grafting reaction can be performed at a pH lower than 6, for instance at a pH from 3.5 to 4.5 as illustrated in Example 2 and described in WO2012090183, the disclosure of which being incorporated herein by reference.

The solid support of the affinity support may be of any type and is selected depending on the contemplated use.

For instance, the solid support may be selected among plastic, metal, and inorganic support such as glass, nickel/nickel oxide, titanium, zirconia, silicon, strained silicon, polycrystalline silicon, silicon dioxide, or ceramic. The said support may be contained in a device such as microelectronic device, microfluidic device, a captor, a biosensor or a chip for instance suitable for use in SPR. Alternatively, the support may be in the form of beads, such as polymeric, metallic or magnetic beads. Such supports may be suitable for detection and diagnostic purposes.

In other embodiments, the solid support may be a polymeric gel, filter or membrane. In particular, the solid support may be composed of agarose, cross-linked agarose, cellulose or synthetic polymers such as polyacrylamide, polyethylene, polyamide, polysulfone, and derivatives thereof. Such supports may be suitable for the purification of IgG. For instance, the solid support may be a support for chromatography, in particular for liquid affinity chromatography. For instance, the affinity support of the invention may be appropriate for carrying out affinity chromatography at the industrial scale. The affinity support of the invention may thus be used as stationary phase in chromatography process, for instance, in column chromatography process or in batch chromatography process.

Uses of the Aptamers and Affinity Ligands According to the Invention in the Purification of IgG and in Other Fields

In an additional aspect, the aptamers and the affinity ligands of the invention may be used in the diagnostic and detection field. In particular, the aptamers and the affinity ligands of the invention may be useful for the diagnostic or the prognostic of diseases or disorders associated with a variation of IgG plasmatic level.

For instance, the aptamers or the ligands of the invention may be used in the diagnostic or the prognostic of disorders such as IgG deficiencies. The aptamers or the ligands of the invention may be used in the diagnostic or the prognostic of disorders wherein the plasma level of IgG is a biomarker of the occurrence or the outcome of the disorders.

In another aspect, the invention relates to a method for capturing IgG, said method comprising:
providing a solid support having an aptamer or an affinity ligand of the invention immobilized thereon,
contacting said solid support with a solution containing IgG, whereby IgG is captured by the formation of a complex between IgG and said aptamer or said affinity ligand immobilized on the solid support.

In some embodiments, the method may comprise one or several additional steps such as:
a step of releasing IgG from said complex,
a step of recovering IgG from said complex
a step of detecting the formation of the complex between IgG and said aptamer or affinity ligand
a step of quantifying IgG,

The detection of the complex and the quantification of IgG (or that of the complex) may be performed by any method known by the skilled artisan. For instance, the detection and the quantification may be performed by SPR as illustrated in the Examples.

Alternatively, one may use an ELISA-type assay wherein a labelled anti-IgG antibody is used for detecting or quantifying the complex formed between IgG and the affinity ligands. The anti-IgG antibody may be labelled with a fluorophore or coupled to an enzyme suitable for the detection, such as the horseradish peroxidase.

The invention also relates to a complex comprising (i) IgG and (ii) an aptamer or an affinity ligand directed to IgG, as described above.

As fully illustrated in Example below, the aptamers of the invention are particularly suitable for a use in the purification of proteins comprising a Fc domain from a IgG such as IgG.

In a particular embodiment, the invention also relates to the use of an aptamer, an affinity ligand or an affinity support of the invention for the purification of IgG. A further object of the invention is thus a method for purifying IgG from a starting composition comprising:
a. contacting said starting composition with an affinity support as defined above, in conditions suitable to form a complex between (i) the aptamers or the affinity ligands immobilized on said support and (ii) IgG,
b. releasing IgG from said complex, and
c. recovering IgG in purified form.

A further object of the invention is a method for preparing a purified IgG composition from a starting IgG-containing composition comprising:
a. contacting said starting composition with an affinity support as defined above, in conditions suitable to form a complex between (i) the aptamers or the affinity ligands immobilized on said support and (ii) IgG,
b. releasing IgG from said complex, and
c. recovering a purified IgG composition.

As used herein, the starting composition may be any composition which potentially comprises IgG, for instance as a single IgG subclass or as a mixture or IgG subclasses. The starting composition may comprise contaminants from which IgG is to be separated.

The contaminants may be of any type and depend on the nature of the starting composition. The contaminants encompass proteins, salts, hormones, vitamins, nutriments, lipids, cell debris such as cell membrane fragments and the like. In some embodiments, the contaminants may comprise blood proteins such as clotting factors, fibronectin, albumin, immunoglobulin, plasminogen alpha-2-macroglobulin and the like.

In some other embodiments, the contaminant may comprise non-human proteins, in particular non-human proteins endogenously expressed by a recombinant host such as a recombinant cell, bacteria or yeast, or a transgenic animal.

Typically, the starting composition may be, or may derive from, a cell culture, a fermentation broth, a cell lysate, a tissue, an organ, or a body fluid.

As used herein, a “starting composition” derives from an entity of interest, such as milk, blood or cell culture, means that the starting composition is obtained from said entity by subjecting said entity to one or several treatment steps. For instance, the entity of interest may be subjected to one or several treatments such as cell lysis, a precipitation step such as salt precipitation, cryo-precipitation or flocculation, a filtration step such as depth filtration or ultrafiltration, centrifugation, clarification, chromatography, an extraction step such as a liquid-liquid or a solid-liquid extraction, viral inactivation, pasteurization, freezing/thawing steps and the like. For instance, a starting composition is derived from blood encompass, without being limited to, plasma, a plasma fraction and a blood cryoprecipitate.

In some embodiments, the starting solution is derived from blood, preferably from human blood. The starting composition may be selected from plasma, plasmatic fraction, for instance fraction II+III obtained by Cohn's ethanol precipitation.

In some other embodiments, the starting composition is obtained from a recombinant host. Preferably, the recombinant host is a transgenic animal, such as a non-human transgenic mammal. The transgenic non-human mammal may be any animal which has been genetically modified so as to express a IgG comprising a Fc domain from a human IgG, such as human IgG, chimeric IgG or humanized IgG. Preferably, said IgG is expressed in a body fluid of said transgenic animal.

The starting solution may thus be, or may derive from, a body fluid of a transgenic animal Body fluids encompass, without being limited to, blood, breast milk, and urine.

In a particular embodiment, the starting composition is, or derives from, milk from a transgenic non-human mammal. Methods for producing a transgenic animal able to secrete a protein of interest in milk are well-known in the state of art. Typically, such methods encompass introducing a genetic construct comprising a gene coding for the protein of interest operably linked to a promoter from a protein which is naturally secreted in milk (such as casein promoter or WHAP promoter) in an embryo of a non-human mammal. The embryo is then transferring in the uterus of a female from the same animal species and which has been hormonally prepared for pregnancy.

In some preferred embodiments, the starting composition may be selected from human blood, transgenic milk and derivatives thereof.

The affinity support used in the methods of the invention may be any affinity supports described hereabove. Preferably, the affinity support is an affinity support for performing affinity chromatography. Indeed, the methods for purifying IgG or preparing a purified composition of IgG are preferably based on chromatography technologies, for instance in batch or column modes, wherein the affinity support plays the role of the stationary phase.

In step a), an appropriate volume of the starting composition containing IgG is contacting with an affinity support in conditions suitable to promote the specific interactions of the anti-IgG aptamer moieties present on the surface of the affinity support with the IgG, whereby a complex is formed between IgG molecules and said aptamer moieties. In step a), IgG is thus retained on the affinity support. The binding between the aptamer moieties and IgG molecules may be enhanced by performing step a) at an acid pH. In some embodiments, step a) is performed at a pH lower than 7, preferably lower than 6.9, 6.8, or 6.7. In particular step a) may be performed at a pH from 4.2 to 6.3, preferably at a pH of 4.5 to 5.7, such as 4.5, 4.6, 4.7, 4.8, 4.9, 5.0, 5.1, 5.2, 5.3, 5.4, 5.5, 5.6, or 5.7. For instance, step a) may be performed at a pH of 5.3 to 5.7 such as a pH of 5.5. In a more general aspect, the pH condition of step a) may be selected so as to promote the binding of IgG onto the affinity support while minimizing the binding of the other molecules onto the affinity support.

Typically, step a) is performed in the presence of a buffer solution (called hereafter a “binding buffer”). The binding buffer can be mixed with the starting composition prior to step a) or can be added during step a). The binding buffer is typically an aqueous solution containing a buffer agent. The buffer agent may be selected so as to be compatible with IgG and the affinity support and so as to obtain the desired pH for step a). For instance, for obtaining a pH of about 5.5 the buffer agent may be selected from, without being limited to, 3-(N-morpholino)propanesulfonic acid (MOPS), 2-(N-morpholino)ethanesulfonic acid (MES), HEPES, Bis-TRIS, citrate and acetate. The buffering agent may be present at a concentration of about 5 mM to 500 mM, for instance from 10 mM to 300 mM such as about 50 mM.

Without to be bound by any theory, the presence of salts may promote the formation of the complex between IgG and the aptamer moieties of the solid support and/or prevent the binding of the other molecules present in the starting composition. Typically, step a) may be performed in the presence of sodium chloride, for instance at a concentration ranging from 10 mM to 500 mM, preferably from 50 mM to 350 mM, or from 100 mM to 200 mM such as about 150 mM. The presence of divalent cations may modulate the binding of IgG to the aptamer moieties. In some embodiments, step a) is performed in the presence of divalent cations, such as Mg²⁺ at a concentration of at least 1 mM, for instance at a concentration of about 1 mM to 50 mM, for instance from 1 mM to 20 mM, such as a concentration of about 5 mM.

In some other embodiments, step a) is performed in the absence of Mg²⁺; and more generally, in the absence of divalent cations.

Accordingly the binding buffer used in step a) may comprise NaCl at a concentration of about 100 mM to 200 mM and magnesium salt such as magnesium chloride (MgCl2) at a concentration of about 1 mM to 50 mM and may have a pH of about 5.5. Such a buffer may be suitable for most of the aptamers of the invention.

An appropriate binding buffer for implementing step a), in particular when the aptamer moiety belongs to the first subgroup as defined above, may be a buffer comprising 50 mM of MES, 5 mM of MgCl₂ and 150 mM of NaCl, at pH 5.5.

Certain aptamers of the invention may work in the absence of Mg²⁺. Thus, in some embodiments, the binding buffer may be devoid of Mg²⁺, and more generally of divalent cations. An appropriate binding buffer for implementing step a) may be thus a buffer comprising 50 mM of MOPS, and 150 mM of NaCl, at pH 5.5.

At the end of step a), and prior to step b), the affinity support may be washed with an appropriate washing buffer so as to remove the substances which are not specifically bound, but adsorbed onto the support. It goes without saying that the washing buffer does not significantly impair the complex between IgG and the aptamer moiety while promoting desorption of the substances which do not specifically bind to the affinity support.

Thus, in some embodiments, the method of the invention comprises a step of washing the affinity support at the end of step a) and before step b). Any conventional washing buffer, well known to those skilled in the art, may be used. In some embodiments, the washing buffer as the same composition as that of the binding buffer used in step a). In other embodiments, the washing buffer may comprise the same components, but at different concentrations, as compared to the binding buffer used in step a). In some additional or alternative embodiments, the pH of the washing buffer is the same as that of the binding buffer.

The washing buffer may have a pH of less than 7, for instance from pH 4.2 to 6.9, preferably from 5.0 to 5.7, such as pH 5.5 The washing buffer may further comprise NaCl. Typically, the ionic strength of the washing buffer may be higher than that of the binding buffer. Indeed, the Applicants showed that, for certain aptamers of the invention, high ionic strength may not significantly impair the binding of IgG to the aptamer moieties. In other words, the complex between IgG and certain aptamers of the invention may be stable, even in the presence of high ionic strength. Thus, in some embodiments, the washing solution has a ionic strength higher than that of the binding buffer used in step a). In alternate or additional embodiments, the washing buffer may comprise a concentration of NaCl of at least 100 mM and up to 10 M. For instance the concentration of NaCl may be of about 100 mM to 5 M, preferably from 150 mM to 2 M. Optionally the washing buffer further comprises divalent cations, in particular Mg²⁺, at a concentration of about 0.1 mM to 20 mM, preferably from 1 mM to 10 mM such as a concentration of about 5 mM. In some embodiments, the washing buffer is devoid of Mg²⁺ and more generally of divalent cations.

In some other or additional embodiments, the washing buffer may comprise at least one additional component, preferably selected among alkyl diols, in particular among ethylene glycol or propylene glycol. Indeed, for certain aptamers of the invention, the presence of alkyl diols such as ethylene glycol in the washing solution do not impair the complex between IgG and the aptamer. The washing buffer may thus comprise an alkyl diol such as ethylene glycol or propylene glycol in an amount from 1% to 70% in weight, preferably from 10% to 60% in weight, such as 50% in weight.

For illustration only, the washing buffer comprises MES at 50 mM, NaCl at 2M, MgCl2 at 5 mM, at pH 5.5 and optionally 50% of glycol in weight.

Step b) aims at releasing IgG from the complex formed in step a). This release may be obtained by destabilizing the complex between IgG and the aptamer moieties, i.e. by using conditions which decrease the affinity of the aptamers to IgG. Noteworthy, the complex between the aptamer moiety and IgG may be destabilized in mild conditions which are not susceptible to alter IgG.

As explained above, the ability of the aptamers of the invention to bind to IgG may depend on the pH of the medium. Increasing the pH above 7.0 may enable to promote the release of IgG. Thus in certain embodiments, step b) is performed by increasing the pH above 7.0. Preferably, the pH of step b) is from 7.0 to 8.0, for instance from 7.2 to 7.8 such as a pH of 7.4. In other words, an elution buffer at pH above 7.0 may be used to promote the release of IgG. A buffer with a pH from 6.5 to 7.0 may be also suitable to promote the elution of IgG.

For illustration only, an appropriate elution buffer may be a buffered solution of 50 mM MES or Tris-HCl at pH 7.4 and comprising 150 mM of NaCl and 5 mM of MgCl₂.

As explained above, the aptamer capability of binding to IgG may also vary depending on the presence of divalent cations, such as Mg²⁺. For instance, the binding of the aptamer moiety to IgG may be promoted by the presence of Mg²⁺. Thus, the release of IgG from the complex in step b) may be promoted by using an elution buffer devoid of divalent cations and/or comprising a divalent cation-chelating agent, such as EDTA or EGTA.

In other embodiments, the binding of the aptamer moiety to IgG may decrease in the presence of divalent cations such as Mg²⁺. Thus, in this embodiment, the elution buffer may comprise divalent cations, in particular Mg²⁺, at a concentration of about 0.1 mM to 20 mM, preferably from 1 mM to 10 mM such as a concentration of about 5 mM.

In some embodiments, the binding buffer used in step a) and the elution buffer used in step b) are devoid of Ca²⁺.

At the end of step c), the purified IgG is typically obtained in the form of a liquid purified composition. This liquid purified composition may undergo one or several addition steps. Said liquid composition may be concentrated, and/or subjected to virus inactivation or removal, for instance by sterile filtration or by a detergent, diafiltration, formulation step with one or several pharmaceutically acceptable excipients, lyophilization, packaging, preferably under sterile conditions, and combinations thereof.

In a more general aspect, the method for purifying IgG or the method for preparing a purified IgG composition may comprise one or several additional steps including, without being limited to, chromatography step(s) such as exclusion chromatography, ion-exchange chromatography, multimodal chromatography, reversed-phase chromatography, hydroxyapatite chromatography, or affinity chromatography, precipitation step, one or several steps of filtration such as depth filtration, ultrafiltration, tangential ultrafiltration, nanofiltration, and reverse osmosis, clarification step, viral inactivation or removal step, sterilization, formulation, freeze-drying, packaging and combinations thereof.

In an additional aspect, the aptamers and the affinity ligands of the invention may be used in a blood plasma fractionation process. The blood plasma fractionation process may comprise several consecutive affinity chromatography steps, each affinity chromatography step enabling to recover a plasma protein of interest such as fibrinogen, immunoglobulin, albumin and other coagulation factors, such as vitamin K-dependent coagulation factors. The affinity ligands used in each step may be of any type, in particular aptamers. To that respect, the Applicant surprisingly showed that plasma proteins such as fibrinogen, albumin, and immunoglobulin, can be recovered and purified from blood plasma by performing successive aptamer-based affinity chromatography steps. Noteworthy, blood plasma fractionation process comprising successive aptamer-based affinity chromatography steps enable to obtain fibrinogen concentrate and immunoglobulin concentrate with a protein purity of at least 96%, and even of at least 99% and with yields of about 9-12 g per plasma litre for immunoglobulins and 2-4 g per plasma litre for fibrinogen. The Applicant further showed that these good yields and purity rates can be achieved from raw blood plasma. In other words, the aptamer-based affinity chromatography steps can be performed on raw blood plasma without any pre-treatment such as ethanol fractionation (Cohn process), cryoprecipitation, caprylate fractionation or PEG precipitation. Notably, such fractionation process enables to avoid temporary intermediary cold storages. Moreover, as shown in Example 3 with the aptamer of SEQ ID NO:22 (core sequence of aptamer A-6.4), the anti-IgG aptamer-based affinity chromatography step may enable to retain the distribution of IgG's subclasses in the elution fraction as compared to the starting composition to purify.

A further object of the invention is thus a blood plasma fractionation process comprising:

(a) an affinity chromatography step to recover fibrinogen wherein the affinity ligand is preferably an aptamer which specifically bind to fibrinogen, and
(b) an affinity chromatography step to recover immunoglobulins of G isotype (IgG) wherein the affinity ligand is an aptamer which specifically bind to IgG, preferably as described herein, wherein the affinity chromatography steps (a) and (b) can be performed in any order.

The affinity chromatography step for recovering fibrinogen can be performed before the affinity chromatography to recover IgG and vice versa. Accordingly, in some embodiments, the blood plasma fractionation process comprises the steps of:
subjecting blood plasma or a derivative thereof to an affinity chromatography step, wherein the affinity ligand is an aptamer which specifically binds to fibrinogen, and
subjecting the non-retained fraction, which is substantially free from fibrinogen, to an affinity chromatography step, wherein the affinity ligand is an aptamer which specifically binds to immunoglobulin of G isotype.

It goes without saying that the above steps may comprise recovering fibrinogen and IgG retained on the affinity support, respectively.

In some other embodiments, the blood plasma fractionation process comprises the steps of:
subjecting blood plasma or a derivative thereof to an affinity chromatography step, wherein the affinity ligand is an aptamer which specifically binds to IgG, and
subjecting the non-retained fraction, which is substantially free from IgG, to an affinity chromatography step, wherein the affinity ligand is an aptamer which specifically binds to fibrinogen.

It goes without saying that the above steps may comprise recovering IgG and fibrinogen retained on the affinity support, respectively.

In the process of the invention, the starting composition can be a blood plasma or derivatives thereof. Derivatives of blood plasma encompass, without being limited to, a clarified blood plasma, a lipid-depleted blood plasma, a blood plasma cryoprecipitate, a supernatant of a blood plasma cryoprecipitate, a plasma fraction and the like. In some embodiments, the starting composition is a raw blood plasma.

Immunoglobulins of G isotype encompass IgG1, IgG2, IgG3 and IgG4. In some embodiments, the aptamer directed against the immunoglobulin of G isotype is able to specifically bind to IgG, regardless IgG subclasses. In some embodiments, several types of anti-IgG aptamers are used so as to recover all the subclasses of IgG. Preferably, the IgG fraction recovered in the fractionation process of the invention has a subclasses distribution close to that of the starting blood plasma, namely comprises from 50% to 70% of IgG1, from 20% to 40% of IgG2, from 2% to 10% of IgG3 and 1 to 8% of IgG4.

In some embodiments, the blood plasma fractionation process of the invention comprises one or several additional steps, in particular (c) a step of purifying albumin.

Purified albumin can be recovered by any conventional methods such as chromatography including affinity chromatography, ion-exchange chromatography, and ethanol precipitation followed by filtration.

For instance, step (c) can be an affinity chromatography step wherein the affinity ligand is an aptamer which specifically bind to albumin

When step (c) is present, steps (a), (b) and (c) can be performed in any order. In some embodiments, step (c) is performed on the non-retained fraction obtained from step (a) or step (b).

Any type of chromatography technology can be used to implement steps (a), (b) and (c) in the process of the invention, such as batch chromatography, Simulated Moving Bed (SMB) Chromatography or Sequential Multicolumn Chromatography (SMCC). Preferred chromatography technologies are those comprising the use of multi-columns such as SMB chromatography and SMCC. Multi-column chromatography technology is based on the use of several small columns, comprising the same stationary phase, instead of one single chromatography column as in the case of batch chromatography. These small columns are typically connected in series.

Examples of multicolumn chromatography process are described for instance in WO2007/144476, WO2009/122281 and WO2015136217, the disclosure of which being incorporated herein by reference.

In some embodiments, the blood plasma fractionation process of the invention comprises at least one multicolumn chromatography step, said step being preferably step (a).

In some other embodiments of the fractionation process of the invention, steps (a) and (b) are multicolumn chromatography steps, in particular SMCC. In some additional or alternate embodiments, step (c) is present and is a multicolumn chromatography step. In some additional steps, all the chromatography steps of the blood plasma process of the invention are multicolumn chromatography steps, in particular SMCC.

In some alternate or additional embodiments, the chromatography column(s) used in steps (a) and/or (b) and/or (c) is/are radial chromatography column(s). Appropriate radial columns encompass, without being limited to, radial columns having a ratio of the largest external diameter surface to the smallest inner diameter surface of 2.

In some embodiments, the binding buffers used in steps (a), (b) and in the optional step (c) are such that the chromatography steps can be performed consecutively, without any pre-treatment steps such as a dialysis or diafiltration step between them. For instance, when step (a) is performed before step (b), the non-retained fraction obtained from step (a) can be used in step (b) without any pre-treatment such as diafiltration.

In some embodiments, the same binding buffer conditions are used in step (a), step (b) and optional step (c). In some other embodiments, the buffers used in steps (a), (b) and in the optional step (c) are such that minor intermediary steps are performed before carrying out the subsequent chromatography step. Minor intermediary steps encompass pH adjustment, conductivity adjustment, and/or ionic strength adjustment of the non-retained fraction resulting from the precedent chromatography step as well as the addition and/or the removal of a specific excipient in said non-retained fraction.

The blood plasma fractionation process can comprise one or several additional steps including, without being limited to, chromatography step to remove anti-A and/or anti-B antibodies, ultrafiltration, tangential ultrafiltration, nanofiltration, reverse osmosis, clarification, viral inactivation step, virus removal step, sterilization, polishing steps such as formulation, or freeze-drying and combinations thereof. The process of the invention may also comprise one or several additional steps aiming at preventing and/or removing the fouling of the chromatography columns such as sanitization with an alkaline solution, e.g. with sodium hydroxide solution.

The invention also relates to a purified composition of IgG obtainable or obtained by a method for preparing a purified IgG composition according to the invention or by the blood plasma fractionation process according to the invention.

A further object of the invention is a purified composition of IgG which comprises at least 90% by weight, preferably at least 91%, 92%, 93%, 94, 95%, 96%, 97% 98%, 99%, 99.1%, 99.2%, 99.3%, 99.4%, 99.5%, 99.6%, 99.7%, 99.8%, or 99.9% by weight as compared to the total weight of proteins present in said composition. In some embodiments, the purified composition of IgG comprises human plasmatic IgG, e.g. IgG obtained from human plasma. In such an embodiment, said composition comprises at most 10%, preferably at most 9%, 8%, 7%, 6%, 5%, 4% 3%, 2%, 1%, 0.9%, 0.8%, 0.7%, 0.6%, 0.5%, 0.4%, 0.3%, 0.2% or 0.1% by weight of other plasma proteins, in particular of other human coagulation factors. In some embodiments, the composition is substantially devoid of human coagulation factors.

In some embodiments, the purified composition of IgG comprises recombinant IgG, e.g. human IgG such as chimeric, humanized or fully human IgG produced in a recombinant host such as recombinant cell or a transgenic animal. In such an embodiment, said composition comprises at most 10%, preferably at most 9%, 8%, 7%, 6%, 5%, 4% 3%, 2%, 1%, 0.9%, 0.8%, 0.7%, 0.6%, 0.5%, 0.4%, 0.3%, 0.2% or 0.1% by weight of other proteins, in particular of non-human proteins from the recombinant host. In some embodiments, the composition is substantially devoid of non-human proteins. In some additional or alternate embodiments, said composition is devoid of any non-human homolog of IgG which may be found in the recombinant host.

The invention also relates to a pharmaceutical composition comprising a purified composition of human IgG such as recombinant human IgG or human plasmatic IgG as defined above, in combination with one or more pharmaceutically acceptable excipients. Said pharmaceutical composition as well as the liquid purified composition of IgG according to the invention can be used in the treatment of coagulation disorders, in particular in the treatments of congenital or acquired deficiency in IgG (hypo-, hyper-, dys- or ahypogammaglobulinemia).

Method for Obtaining the Aptamers of the Invention

The Applicants carried-out several SELEX strategies described in the prior art to identify aptamers against human IgG. None of these strategies succeeded in the identification of an aptamer against a common region of IgGs. For example, a standard SELEX performed on the Fc fragment derived from a monoclonal IgG led to the identification of aptamers against the hypervariable region of the monoclonal IgG, which was present in trace amounts in the Fc preparation.

In that context, the Applicant performed extensive researches to develop a new method for obtaining aptamers directed against “SELEX-resistant” proteins such as IgG.

The Applicant conceived a new SELEX process which enables to obtain aptamers displaying high binding affinity for “SELEX-resistant” proteins, and which may be used as affinity ligands in purification process. This new SELEX process is characterized by a selection step which is performed in conditions of pH suitable to create “positive patches” on the surface of the protein target. In other words, the process conceived by the Applicant is based on the enhancement of the local interactions between the potential aptamers and the targeted protein by promoting positive charges on a surface domain of the protein. This method can be implemented for proteins having one or several surface histidines, such as IgG. The pH of the selection step (i.e. the step wherein the protein target is contacted with the candidate mixture of nucleic acids) should be selected so as to promote the protonation of at least one surface histidine of the protein target. In the case of IgG, the applicant showed that an appropriate pH for the selection step is an acid pH.

Accordingly, the invention also relates to a method for obtaining an aptamer which specifically binds to IgG on its Fc region, said method comprising:
a) contacting Fc fragment of IgG (IgG-Fc) with a candidate mixture of nucleic acids at a pH lower than 7.0, preferably from 4.2 to 5.7,
b) recovering nucleic acids which bind to IgG-Fc, while removing unbound nucleic acids,
c) amplifying the nucleic acids obtained in step (b) to yield to a candidate mixture of nucleic acids with increased affinity to IgG-Fc, and
d) repeated steps (a), (b), (c) until obtaining one or several aptamers against IgG-Fc.

In step (a), the candidate mixture of nucleic acids is generally a mixture of chemically synthesized random nucleic acid. The candidate mixture may comprise from 10⁸ to 10¹⁸, typically about 10¹⁵ nucleic acids. The candidate mixture may be a mixture of DNA nucleic acids or a mixture of RNA nucleic acids. In some embodiments, the candidate mixture consists of a multitude of single-stranded DNAs (ssDNA), wherein each ssDNA comprises a central random sequence of about 20 to 100 nucleotides flanked by specific sequences of about 15 to 40 nucleotides which function as primers for PCR amplification. In some other embodiments, the candidate mixture consists of a multitude of RNA nucleic acids, wherein each RNA comprises a central random sequence of about 20 to 100 nucleotides flanked by primer sequences of about 15 to 40 nucleotides for RT-PCR amplification. In some embodiments, the candidate mixture of nucleic acids consists of unmodified nucleic acids, this means that the nucleic acids comprise naturally-occurring nucleotides only. In some other embodiments, the candidate mixture may comprise chemically-modified nucleic acids. In other words, the nucleic acids may comprise one or several chemically-modified nucleotides. In preferred embodiments, the candidate mixture consists of single-stranded DNAs.

Step a) is performed in conditions favourable for the binding of IgG-Fc with nucleic acids having affinity for said IgG. Preferably, the pH of step a) is from 5.0 to 5.7, such as 5.1, 5.2, 5.3, 5.4 5.5 and 5.6. An appropriate pH for step a) is for instance, 5.5±0.1. Such pH enables to protonate at least one surface histidine of IgG. Step (a) may be performed in a buffered aqueous solution. The buffering agent may be selected from any buffer agents enabling to obtain the desired pH and compatible with the protein targets and the nucleic acids mixture. The buffer agent may be selected from, without being limited to, 3-(N-morpholino)propanesulfonic acid (MOPS), 2-(N-morpholino)ethanesulfonic acid (MES), HEPES, Bis-TRIS, citrate and acetate. The buffering agent may be present at a concentration of about 5 mM to 1 M, for instance from 10 mM to 500 mM, for instance from 10 mM to 200 mM such as about 50 mM.

In some embodiments, IgG-Fc may be present in free-state in step (a). In some other embodiments, IgG-Fc may be immobilized on a solid support in order to make easier the subsequent separation of the complex formed by the protein target with certain nucleic acids and the unbound nucleic acids in step (b). For instance, IgG-Fc may be immobilized onto magnetic beads, on solid support for chromatography such as sepharose or agarose, on microplate wells and the like. Alternatively, IgG-Fc may be tagged with molecules useful for capturing of the complex in step (b). For instance, IgG-Fc may be biotinylated.

Step (b) aims at recovering nucleic acids which bind to IgG-Fc in step (a), while removing unbound nucleic acids. Typically, step (b) comprises separating the complex formed in step (a) from unbound nucleotides, and then releasing the nucleic acids from the complex whereby a new mixture of nucleic acids with increased affinity to the target protein is obtained.

The separation of the complex from the unbound nucleic acids may be performed by various methods and may depend on the features of IgG-Fc. These methods include without being limited to, affinity chromatography, capillary electrophoresis, flow cytometry, electrophoretic mobility shift, Surface Plasmon resonance (SPR), centrifugation, ultrafiltration and the like. The skilled artisan may refer to any separation methods described in the state in the art for SELEX processes, and for instance described in Stoltenburg et al. Biomolecular Engineering, 2007, 24, 381-403, the disclosure of which being incorporating herein by reference. As illustration only, if IgG is immobilized on a support, the separation may be performed by recovering the support, washing the support with an appropriate solution and then releasing nucleic acids from the complex immobilized on the support. If IgG-Fc has been incubated in free-state with the candidate mixture, the separation of the nucleic acid-protein complex from unbound nucleic acids can be performed by chromatography by using a stationary support able to specifically bind to fibrinogen or the possible tag introduced on IgG-Fc, whereby the complexes are retained on the support and the unbound nucleic acids flow out. For instance, one may use a stationary phase having thereon antibodies directed against the target protein. Alternatively, the partitioning may be performed by ultrafiltration on nitrocellulose filters with appropriate molecular weight cut-offs. Once the complexes separated from unbound nucleic acids, the nucleic acids which bind to IgG are released from the complexes. The release can be performed by denaturing treatments such as heat treatment or by elution. Preferably, said nucleic acids are recovered by using an elution buffer able to dissociate the complex. The dissociation may occur by increasing the ionic strength or by modulating the pH in the elution buffer as compared to the buffered solution used in step a). For instance, if the pH of step (a) is 5.5, the pH of the elution buffer may be from 6.5 to 7.9, such as 7.4.

In a particular embodiment, step b) comprises the steps of separating the complex formed in step (a) from unbound nucleic acids, and then releasing the bound nucleic acids from the complex. The dissociation of the complex between IgG-Fc and bound nucleic acids can be performed by increasing the pH above 7.0 in step b). Typically, in step b) the nucleic acids are recovered by dissociating the complex between IgG and the nucleic acids at a pH above 7.0, for instance from pH 7.0 to 8.0, preferably from pH 7.2 to 7.8, more preferably from 7.2 to 7.6, such as 7.4. Preferably, in step b), the complex is immobilized on a solid support by the mean of IgG. This means that IgG-Fc is immobilized by covalent or non-covalent interactions on the solid support as described above. After an optional washing step, typically with the buffer used in step a), the complex between the nucleic acids and IgG-Fc can be dissociated with an elution buffer having a pH from pH 7.0 to 8.0, preferably from pH 7.2 to 7.8, more preferably from 7.2 to 7.6, such as 7.4. The nucleic acids of interest are thus recovered in the elution buffer.

In alternate or additional embodiments, the elution buffer may comprise EDTA or detergent such as SDS, or urea. For instance, the elution buffer may comprise EDTA at a concentration of about 100 mM to 500 mM.

In step (c), the nucleic acids recovered in step (b) are amplified so as to generate a new mixture of nucleic acids. This new mixture is characterized by an increased affinity to the target protein as compared to the starting candidate mixture.

Step (a), (b) and (c) form together a round of selection. As indicated in step (d), this round of selection can be repeated several times, typically 6-20 times until obtaining an aptamer or a pool of aptamers directed against the target protein. It goes without saying that the step (a) of round “N” is performed with the mixture of nucleic acids obtained in step (c) of the round “N−1”. At the end of each selection round, the complexity of the mixture obtained in step (c) is reduced and the enrichment in nucleic acids which specifically bind to the target protein is increased.

The conditions for implementing step (a), (b) and (c) may be the same or may be different from one round of selection to another. In particular, the conditions of step (a) (e.g. the incubation conditions of the target protein with the mixture of nucleic acids) can change. For instance, step (a) of round “N” can be performed in more drastic conditions than in round “N+1” in order to direct the selection to aptamers having the highest affinity for IgG. Typically, such result can be obtained by increasing the ionic strength of the buffer used in step (a).

The method of the invention may comprise one or several additional steps. The method of the invention may comprise counter-selection or substractive selection rounds. The counter-selection rounds may aim at eliminating nucleic acids which cross-react with other entities or directing the selection to aptamers binding to a specific epitope of Fc-IgG.

The method of the invention may comprise one or several of the following steps:
a step of cloning the aptamer pool,
a step of sequencing an aptamer,
a step of producing an aptamer, for instance by chemical synthesis,
a step of identifying consensus sequences in the pool of aptamers, for instance by sequence alignment,
a step of optimizing the sequence of an aptamer,

In some embodiments, the method of the invention may comprise the following additional steps:
sequencing an aptamer obtained in step (c)
optimizing said aptamer, and
producing the optimized aptamer, preferably by chemical synthesis.

The optimization of the aptamer may comprise the determination of the core sequence of the aptamer, i.e. the determination of the minimal nucleotide moiety able to specifically bind to IgG. Typically, truncated versions of the aptamer are prepared so as to determine the regions which are not important in the direct interaction with IgG.

The binding capacity of the starting aptamer and the truncated versions may be assessed by any appropriate methods such as SPR.

Alternatively or additionally, the sequence of the aptamer may be subjected to mutagenesis in order to obtain aptamer mutants, for instance with improved affinity or specificity as compared to their parent aptamer. Typically one or several nucleotide modifications are introduced in the sequence of the aptamer. The resulting mutants are then tested for their ability to specifically bind to IgG, for example by SPR or ELISA-type assay.

In additional or alternate embodiments, the optimization may comprise introducing one or several chemical modifications in the aptamer. Typically, such modifications encompass replacing nucleotide(s) of the aptamer by corresponding chemically-modified nucleotides. The modifications may be performed in order to increase the stability of the aptamers or to introduce chemical moiety enabling functionalization or immobilization on a support.

Further aspects and advantages of the present invention are disclosed in the following experimental section, which should be regarded as illustrative and not limiting the scope of the present application.

### EXAMPLES

### Example 1: Identification of Anti-IgG Aptamers by the Method of the Invention

### 1. Material and Method

Oligonucleotide Library

The ssDNA library used in the SELEX process of the invention consisted of a 40-base random region flanked by two constant 18-base primer regions.

Human Polyclonal IgG-Fc Fragments

The protein target used for the SELEX was highly pure human polyclonal IgG, Fc fragment. It was obtained from Jackson ImmunoResearch Laboratories, INC (ref. 009-000-008).

SELEX Protocol

During the course of the SELEX, continuously decreasing amounts of highly pure human IgG, Fc fragment was incubated with the ssDNA library/pool at decreasing concentrations using as selection buffer 50 mM MES pH 5.50, 150 mM NaCl, 5 mM MgCl2 at decreasing incubation times (see table).

The unbound ssDNA was partitioned from IgG-Fc/ssDNA complexes using nitrocellulose filters. The complex containing filters were washed with selection buffer during round 1, 2, & 3 and wash buffer containing 50 mM MOPS pH 5.50, 500 mM NaCl, 5 mM MgCl2 during round 4 to 6 and wash buffer containing 50 mM MOPS pH 5.50, 1M NaCl, 5 mM MgCl2 during round 7 & 8 (see table). After washing, the bound ssDNA was eluted using elution buffer (50 mM Tris-HCl pH 7.40, 200 mM EDTA).

Before every round (except the first round) a counter selection step was performed by incubating the ssDNA pool with one nitrocellulose filter in order to prevent the enrichment of anti-nitrocellulose aptamers.

The parameters of the SELEX protocols are depicted in FIG. 1.

Determination of the Binding Affinity of the Identified Aptamers by SPR:

The selected aptamer was synthetized with Biotin and a triethylene glycol spacer at the 5′ end of the oligonucleotide. A 1 μM solution of the aptamer was prepared using the SELEX selection buffer. The aptamer solution was heated to 90° C. for 5 min, incubated on ice for 5 min and equilibrated to room temperature for 10 min. The preparation was injected on a streptavidin coated sensor chip SA of Biacore T200 instrument (GE Healthcare) at a flow rate of 10 μl/min for 7 min. Then, different concentrations of the target (Human polyclonal IgG, purified from human plasma with a purity of >95%) were injected to the immobilised aptamer at 30 μl/min for 1 minute. After dissociation for 1-2 min a wash step was performed by injecting a suitable wash buffer at 30 μl/min for 1 min. For elution, a suitable elution buffer was injected at 30 μl/min for 1-2 min Finally the sensor chip was regenerated by injection of 50 mM NaOH at 30 μl/min for 30 sec. During the course of the experiment the response signal was recorded in a sensorgram.

### 2. Results

The SELEX method of the invention enables to identify several anti-IgG aptamer candidates, among which aptamers of SEQ ID NO:1 and SEQ ID NO:2 both flanked by their primers of SEQ ID NO: 19 and SEQ ID NO: 20. The binding ability of these aptamers to polyclonal IgG was assessed by SPR.

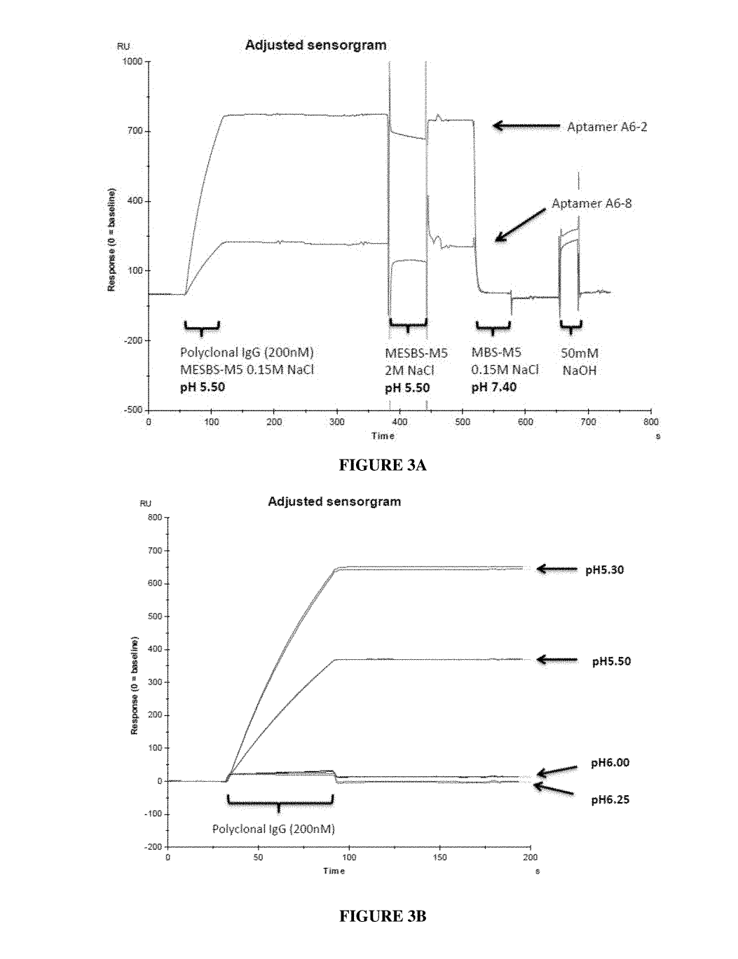

FIG. 3A shows the binding curves of human polyclonal IgG for aptamers A6-2 and A6-8 (namely an aptamer of formula (A) wherein X is SEQ ID NO: 1 or SEQ ID NO: 2 respectively), immobilized on a sensor chip. The aptamers were shown to bind to polyclonal IgG at pH 5.5. The injection of a buffer solution at pH 5.50 comprising 2M NaCl did not significantly induce the elution of human polyclonal IgG. The complex between the aptamers and polyclonal IgG was dissociated by increasing the pH of the buffer. Human polyclonal IgG was then released from the complex by an elution buffer at pH 7.40. The anti-IgG aptamers of the invention specifically bound to their target protein in a pH-dependent manner. The highest binding was obtained for pH 5.30. The binding level decreased, with the increase of pH. No significant binding was observed for pH higher than pH 6.0 (FIG. 3B).

The affinity of aptamers A6-2, A6-8, A6-4 (aptamer of formula (A) with [X] is SEQ ID NO:7) and A6-3 (aptamer of formula (A) with [X] is SEQ ID NO:11) for the different plasma IgG's sub-classes was evaluated by SPR.

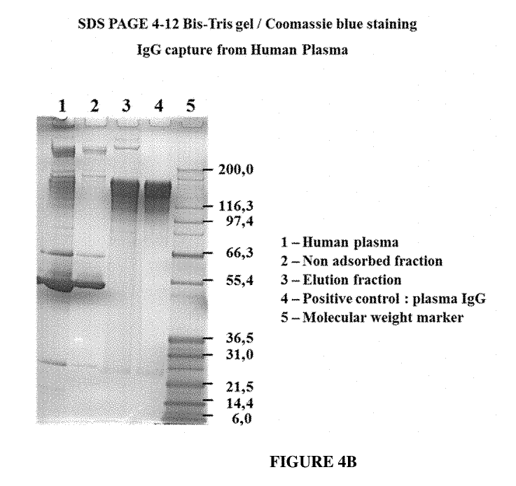

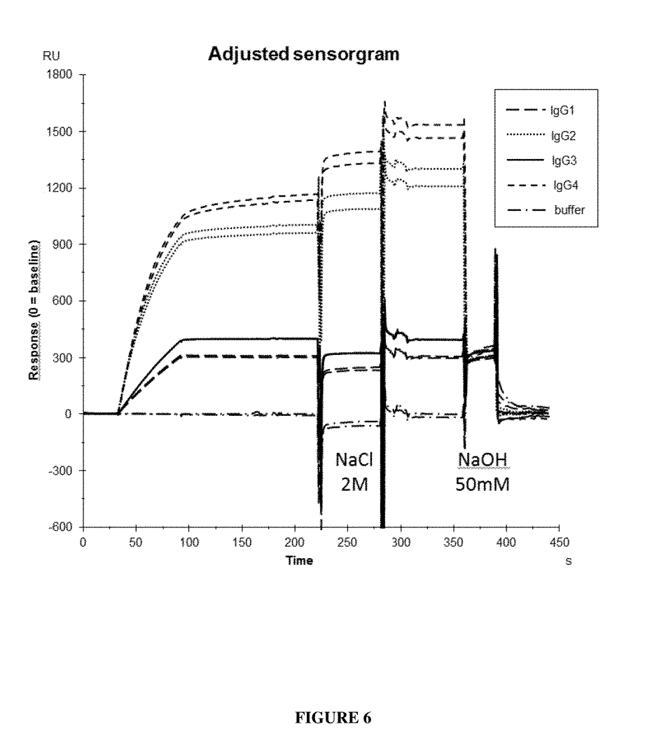

First subgroup members namely aptamers A6-2 and A6-8 show the formation of a complex during the injection of each IgG's sub-class (1, 2, 3 and 4) with varying association rates (FIGS. 5 and 7). The formed aptamer-IgG complexes were resistant to high salt wash (2M NaCl) for IgG's sub-classes 1, 2, and 4, while the aptamer IgG's sub-class 3 complex was less resistant.

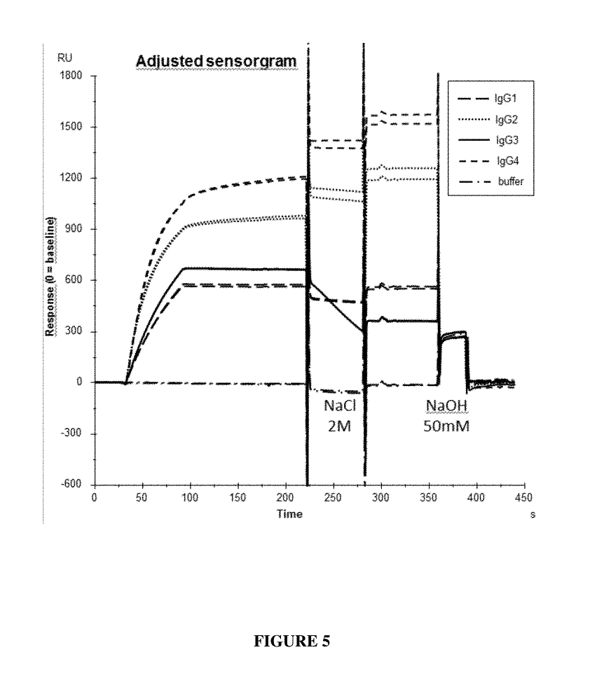

Second subgroup member namely aptamer A6-4 shows as well the formation of a complex during the injection of each IgG's sub-class (1, 2, 3 and 4) with varying association rates (FIG. 6). The resulting aptamer complexes with IgG's sub-class 1, 2, 3 and 4 show a strong resistance to high salt wash (2M NaCl).

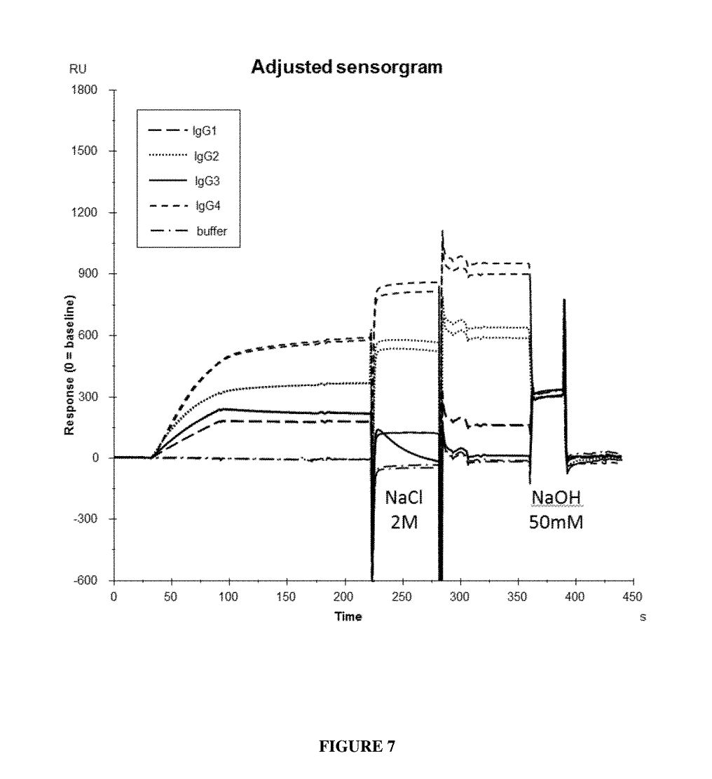

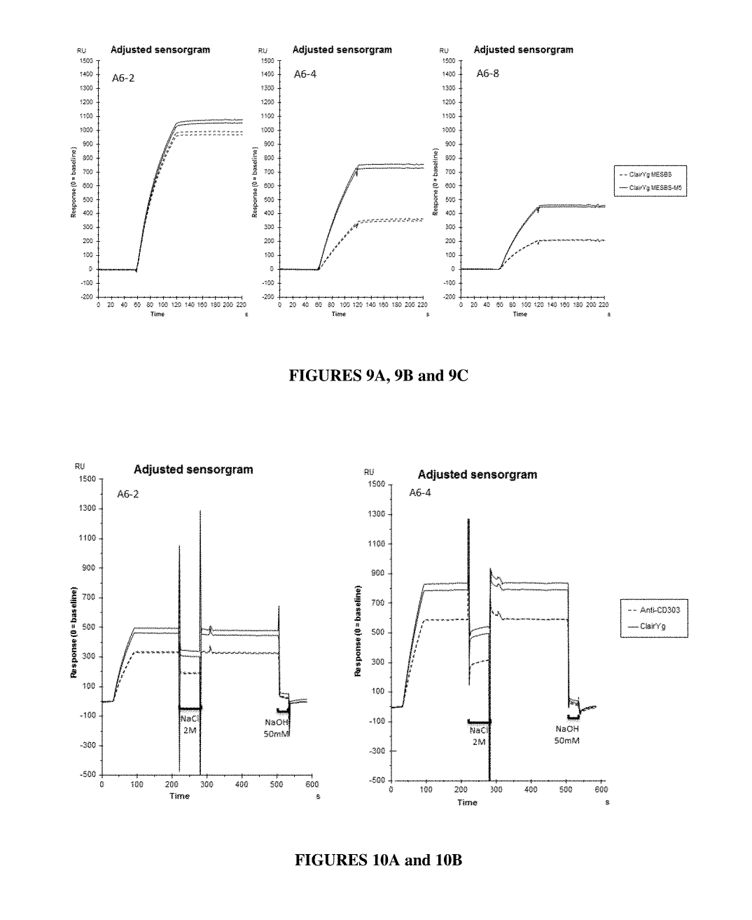

Third subclass member namely aptamer A6-3 shows a considerably faster association rate for sub-classes 2 and 4 than for 1 and 3 (FIG. 8). The injection of a buffer solution at pH 5.50 comprising 2M NaCl did significantly induce the elution of human plasma IgG's sub-classes 1 and 3, and to some extend IgG4, leaving only IgG's sub-classes 2 resistant to 2M NaCl washes. The affinity of aptamers A6-2 and A6-4 for recombinantly produced IgG was evaluated by SPR (FIGS. 10A and 10B). The resulting binding profiles were similar to those obtained for human polyclonal IgG sample. The complexes of aptamer with the recombinant IgG were resistant to high stringency salt washes. Therefore, the aptamers of the invention are expected to be applicable for the purification of recombinantly produced IgG.

### Example 2: Affinity Support and Purification of IgG from Plasma

1. Material and Method

Affinity Support

An affinity support was prepared by grafting aptamer A6-2 comprising a C6 spacer with a terminal amino group at its 5′ end and inverted deoxy-thymidine at its 3′ end on NHS-activated Sepharose (GE Healthcare):

1 volume of NHS Sepharose activated gel placed in a column was rinsed with at least 10 volumes of a cold 0.1M HCl solution, then equilibrated with at least 8 volumes of cold 100 mM acetate pH 4.0 solution.

After a 3 min-2000 g centrifugation, the supernatant is removed and drained gel is re-suspended with 2 volumes of an aptamer in 100 mM acetate pH 7.0 solution. This suspension is incubated at room temperature under stirring.

After 2 hours, 1 volume of 200 mM Borate pH 9 is added. This suspension is incubated at room temperature under stirring for 2H30.

After a 3 min-2000 g centrifugation, the supernatant is removed. Drained gel is re-suspended in 2 volumes of 0.1M Tris-HCl pH 8.5 solution. Suspension is incubated at +4° C. under stirring overnight.

After incubation, and a 3 min-2000 g centrifugation, the supernatant is removed. The gel alternatively washed with 2 volumes of 0.1M Sodium acetate+0.5M NaCl pH 4.2 and 2 volumes of a 0.1M Tris-HCl pH 8.5 solution. This cycle is repeated once.

After a 3 min-2000 g centrifugation supernatant is removed. Drained gel is re-suspended in 2 volumes of binding buffer.

4 mg of aptamer A6-2 was used to be grafted on 1 ml of resine.

Purification of Polyclonal IgG from Purified Plasma IgG or from Plasma

1.1 ml of affinity support was packed in a Tricorn 5/50 column (GE Heathcare). Purified plasma IgG or plasma were diluted with binding buffer to reach a 0.8-1 g/L IgG in final concentration. The pH was then adjusted to 5.5 with 1M citric acid and then filtered 0.45 μm before loading onto the column. Chromatography buffers are described in the following table.

| | Affinity support grafted with Aptamer A6-2 |
|---|---|
| Binding buffer | Buffering agent: MES 50 mM |
| | NaCl 150 mM, MgCl₂ 5 mM, pH 5.5 |
| Elution buffer | Buffering agent: MES 50 mM |
| | NaCl 150 mM, MgCl₂ 5 mM, pH 7.4 |

The linear flow rate used for the chromatography was 100 cm/h, and the quantity of IgG loaded was targeted to be close to the resin capacity (6.5 g/L of resin).

2. Results

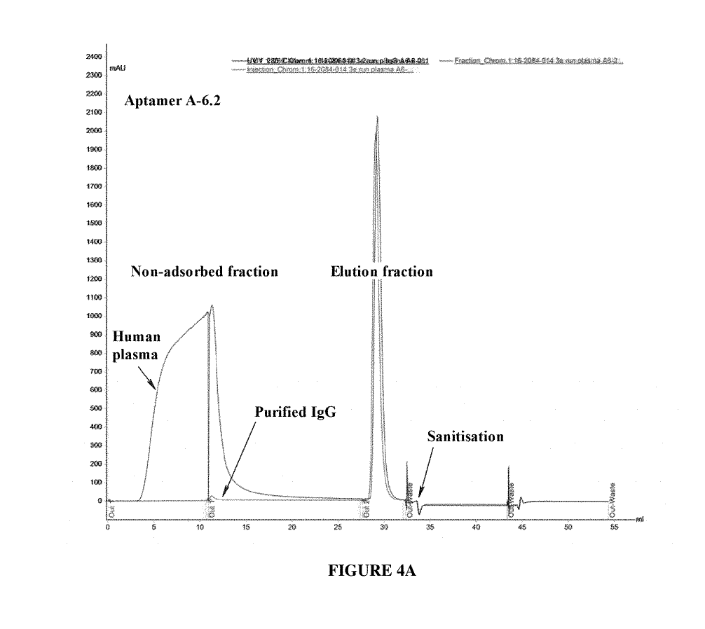

The results are shown in FIGS. 4A-4B. FIG. 4A shows the chromatography profile obtained for the IgG from plasma and pre-purified plasma IgG on an affinity support grafted with aptamer A6-2. Noteworthy, most of the contaminant proteins were not retained on the stationary phase whereas IgG bound to the support. IgGs were eluted by increasing the pH to 7.4. FIG. 4B shows the analysis by SDS Page of the fractions obtained by chromatography for plasma as starting solution. IgGs were mostly present in the elution fraction (lane 3) whereas contaminant proteins were present in the non-retained fraction (lane 2). The relative purity of the IgG eluted from the affinity column was more than 95% by SDS-PAGE. The high purity of the elution fraction demonstrated the high specificity of the aptamer for IgG. The yield of the chromatography was 82% from pre-purified IgGs and 66% from plasma. Yield could be increased with loading a quantity of IgG bellow the capacity of the resin. The aptamers identified by the method of the invention thus have binding properties suitable for use in protein purification.

| Purification with | | | |
|---|---|---|---|
| aptamer of SEQ ID | | | |
| NO: 1 (A6-2) flanked | | | |
| by SEQ ID NO: 19 | Quantity of IgG in | Quantity of IgG in | |
| and SEQ ID NO: 20 | the loaded material | the eluate | Yield |
| Purified IgG | 6.3 mg | 5.2 mg | 82% |
| Plasma | 7.9 mg | 5.2 mg | 66% |

### Example 3: Assessment of Aptamer of SEQ ID NO:22 for the Purification of Human Plasmatic IgG

1. Material and Method

Affinity Support

An affinity support was prepared by grafting the aptamer of SEQ ID NO:22 (core sequence of aptamer A6.4) comprising a C6 spacer with a terminal amino group at its 5′ end on NHS-activated Sepharose (GE Healthcare), according to a protocol similar to that used in Example 2 for the grafting of aptamer A6-2 and with amounts appropriate to obtain an aptamer density of 4 mg per ml of gel.

1 mL of gel was prepared accordingly.

Purification of Polyclonal IgG from Purified Plasma IgG or from Plasma

0.9 ml of affinity gel was packed in a Tricorn 5/50 column (GE Heathcare).

Chromatography buffers are described in the following table.

| | Affinity support grafted with Aptamer A6-4 |
|---|---|
| Binding buffer | Buffering agent: MES 50 mM |
| | NaCl 150 mM, MgCl₂ 5 mM, pH 5.5 |
| Elution buffer | Buffering agent: MES 50 mM |
| | NaCl 150 mM, MgCl₂ 5 mM, pH 7.4 |
| Sanitisation buffer | Urea 6M, citric acid 0.2M, pH 3 |

The composition to purify (namely plasma and pre-purified IgG) was diluted in MES buffer containing 5 mM of MgCl₂. The pH was adjusted at pH 5.5.

Two assays were performed with purified IgG with the following load: 25 g of IgG/L of gel and 8 g of IgG per L of gel. The load the plasma was 8 g of IgG per L of gel.

2. Results

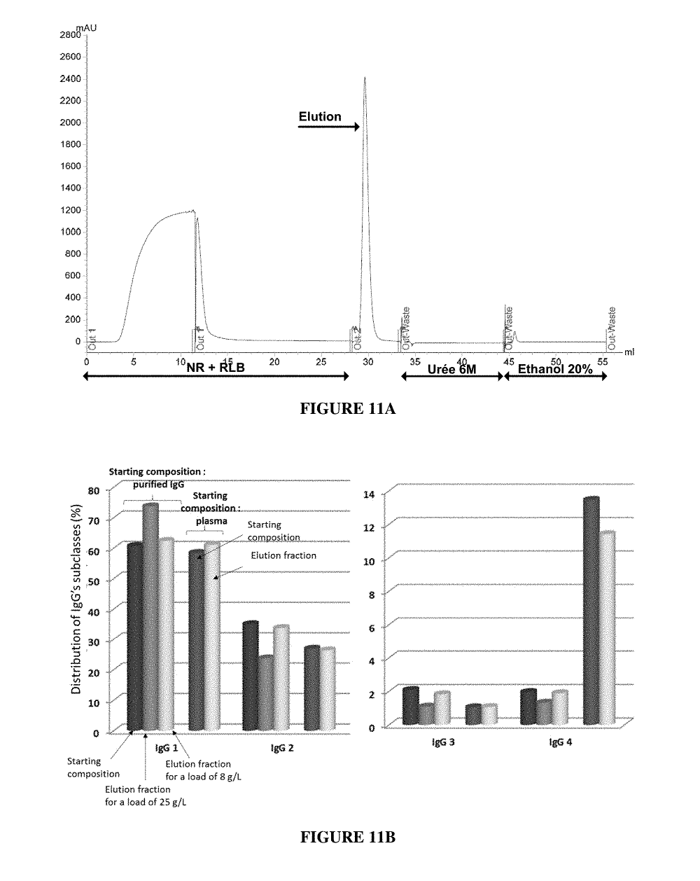

The results are shown in FIGS. 11A and 11B. FIG. 11A shows the chromatography profile obtained for plasma on an affinity support grafted with aptamer of SEQ ID NO:22. Noteworthy, most of the contaminant proteins were not retained on the stationary phase whereas IgG bound to the support. IgGs were eluted by increasing the pH to 7.4. Noteworthy, the sanitisation did not lead to the elution of any additional IgG, which shows the efficacy of the elution buffer. FIG. 11B shows the distribution of IgG's subclasses obtained in the different elution fractions as compared to the starting compositions. Noteworthy, we can note that the chromatography step with aptamer of SEQ ID NO:22 did not significantly impair the IgG's subclasses distribution, especially when the starting composition was plasma or pre-purified IgG with a load of 8 g of IgG/L of gel: the proportions of each IgG subclass was retained in the elution fractions as compared to the starting composition.

Table of sequences

| NO of SEQ ID | Description |
|---|---|
| 1-3 | Central regions of aptamers of the first subgroup |
| 4-8 | Central regions of aptamers of the second subgroup |
| 9-15 | Central regions of aptamers of the third subgroup |
| 16 | Consensus sequence of the first subgroup of aptamers |
| 17 | Consensus sequence of the second subgroup of aptamers |
| 18 | Consensus sequence of the third subgroup of aptamers |
| 19 | First primer sequence |
| 20 | Second primer sequence |
| 21 | Core sequence of the aptamer A6-2 |
| 22 | Core sequence of the aptamer A6-4 |
| 23 | Core sequence of the aptamer A6-8 |

### Structure of Aptamers A6-2, A6-8, A6-3 and A6-4

5′-[SEQ ID NO:19]-[X]-[SEQ ID NO:20]-3′ (A)

Wherein:

For A6-2, [X] is SEQ ID NO:1,

For A6-8, [X] is SEQ ID NO:2,

For A6-3, [X] is SEQ ID NO:11, and

For A6-4, [X] is SEQ ID NO:7

1.-15. (canceled)

16. An aptamer that specifically binds to at least 2 subclasses of human IgG selected from the group consisting of IgG1, IgG2, IgG3, and IgG4, wherein the aptamer does not bind to IgG at a pH higher than 6.5, and wherein the aptamer binds to IgG at an acidic pH below 6.5.

17. The aptamer of claim 16, wherein the aptamer specifically binds to IgG1, IgG2, IgG3, and IgG4

18. An aptamer capable of specifically binding to human IgG, wherein the aptamer comprises a moiety selected from the group consisting of SEQ ID NO:16, SEQ ID NO:17, and SEQ ID NO:18, or that differs from a moiety selected from the group of SEQ ID NO:16, SEQ ID NO:17, and SEQ ID NO: 18 due to 1, 2, 3, 4, or 5 nucleotide modifications.

19. The aptamer of claim 18, wherein the aptamer comprises a polynucleotide:
having at least 70% identity with a sequence selected from the group consisting of SEQ ID NO: 1-15, and SEQ ID NO:21-23, and
comprising a moiety selected from SEQ ID NO:16, SEQ ID NO:17, and SEQ ID NO:18, or that differs from a moiety selected from the group consisting of SEQ ID NO:16, SEQ ID NO:17, and SEQ ID NO: 18 due to 1, 2, 3, 4, or 5 nucleotide modifications.

20. The aptamer of claim 18, wherein the aptamer is capable of specifically binding to IgG, and wherein the aptamer comprises
5′-[NUC 1]m-[CENTRAL]-[NUC2] n−3′
wherein:
n and m are integers independently selected from 0 and 1,
[NUC1] is a polynucleotide comprising from 2 to 40 nucleotides,
[NUC2] is a polynucleotide comprising from 2 to 40 nucleotides, and
[CENTRAL] is a polynucleotide having at least 70% of sequence identity with a nucleotide sequence selected from the group consisting of SEQ ID NO 1-15 and/or comprising a polynucleotide selected from the group consisting of SEQ ID NO: 16, SEQ ID NO: 17 and SEQ ID NO: 18.

21. The aptamer of claim 20, wherein [NUC1] comprises SEQ ID NO: 19, or which differs from SEQ ID NO: 19 due to 1, 2, 3, 4, or 5 nucleotide modifications, and [NUC2] comprises SEQ ID NO:20, or which differs from SEQ ID NO:20 due to 1, 2, 3, 4, or 5 nucleotide modifications.

22. The aptamer of claim 20, wherein [CENTRAL] is SEQ ID NO: 1-15 or differs from SEQ ID NO: 1-15 due to 1, 2, 3, 4, 5, 6, 7, 8, or 9 nucleotide modifications.

23. The aptamer of claim 18, wherein the aptamer is of formula (A):
5′-[SEQ ID NO:19]-[X]-[SEQ ID NO:20]-3′ (A)
wherein:
[SEQ ID NO:19] refers to SEQ ID NO:19,
[SEQ ID NO:20] refers to SEQ ID NO:20, and
[X] is a polynucleotide selected from the group consisting of SEQ ID NO:1-15.

24. The aptamer of claim 18, wherein the aptamer specifically binds to human plasma IgG or recombinant human IgG.

25. The aptamer of claim 18, wherein the aptamer specifically binds to human plasma IgG or recombinant human IgG

26. An affinity ligand capable of specifically binding IgG which comprises an aptamer moiety according to claim 18 and at least one moiety selected from a mean of detection and a mean of immobilization onto a support.

27. A solid affinity support comprising thereon a plurality of aptamers according to claim 18.

28. A solid affinity support comprising thereon a plurality of aptamers according to claim 16.

29. A method for preparing a purified IgG composition from a starting IgG-containing composition, comprising:
a) contacting said starting composition with an affinity support according to claim 16, in conditions suitable to form a complex between (i) the aptamers or the affinity ligands immobilized on said support and (ii) IgG;
b) releasing IgG from said complex; and
c) recovering a purified IgG composition.

30. The method of claim 29, wherein step a) is performed at a pH lower than 7.0, and step b) is performed at a pH above 7.0.

31. The method of claim 29, wherein steps a) to c) are performed using column or batch chromatography.

32. A method for preparing a purified IgG composition from a starting IgG-containing composition comprising:
a) contacting said starting composition with an affinity support as defined in claim 18, in conditions suitable to form a complex between (i) the aptamers or the affinity ligands immobilized on said support and (ii) IgG;
b) releasing IgG from said complex; and
c) recovering a purified IgG composition.

33. A method of purification of IgG, the detection of IgG, or blood plasma fractionation, comprising use of the aptamer of claim 16.

34. A blood plasma fractionation process comprising the following steps in any order:
(a) an affinity chromatography step to recover fibrinogen, wherein the affinity ligand specifically binds to fibrinogen,
(b) an affinity chromatography step to recover immunoglobulins of G isotype (IgG), wherein the affinity ligand is an aptamer which specifically bind to IgG as defined in claim 16, and
(c) optionally a purification step of albumin.

35. A blood plasma fractionation process comprising the following steps in any order:
(a) an affinity chromatography step to recover fibrinogen wherein the affinity ligand specifically binds to fibrinogen,
(b) an affinity chromatography step to recover immunoglobulins of G isotype (IgG) wherein the affinity ligand is an aptamer which specifically bind to IgG as defined in claim 18, and
(c) optionally a purification step of albumin.


SEQUENCE LISTING (23 sequences)

SEQ ID NO: 1 - DNA, 39 bp, Artificial
  Central sequence
cccagcctcatctcacggcatagtctcgccacactggaa

SEQ ID NO: 2 - DNA, 40 bp, Artificial
  Central sequence
cacggtatagtctcgcccagtgccctttgttggacttcct

SEQ ID NO: 3 - DNA, 40 bp, Artificial
  central sequence
ttccacggtatagtctcgccaggttccttgttgacattcc

SEQ ID NO: 4 - DNA, 40 bp, Artificial
  central sequence
tgctaatggtccaggggctggggtgtggtcctggcggacc

SEQ ID NO: 5 - DNA, 39 bp, Artificial
  central sequence
aaggcaatgggcaggggctggggggtggttctggcgctc

SEQ ID NO: 6 - DNA, 40 bp, Artificial
  central sequence
gagaggggctggggggtggttctggccgcagagagaggga

SEQ ID NO: 7 - DNA, 40 bp, Artificial
  central sequence
aagtttgtggggctggggttcggtcctggcacaaattcgt

SEQ ID NO: 8 - DNA, 40 bp, Artificial
  central sequence
gggttatggtgtggcgtgggggtcagctggtatttgccgg

SEQ ID NO: 9 - DNA, 40 bp, Artificial
  central region
acgtcccaccggcctctgcgctcgtcccctaatcagtggc

SEQ ID NO: 10 - DNA, 40 bp, Artificial
  central region
cccgccggcttcctatactttccctaatcagtggcatcgg

SEQ ID NO: 11 - DNA, 40 bp, Artificial
  central region
gactattcctccggcctcttccctaatcagtggcatctct

SEQ ID NO: 12 - DNA, 40 bp, Artificial
  central sequence
ctatttgccgaacctccggcctctctccctaatcagtggc

SEQ ID NO: 13 - DNA, 40 bp, Artificial
  central sequence
ctttccctccggcctctcccctaatcagtggtgttggccc

SEQ ID NO: 14 - DNA, 40 bp, Artificial
  central sequence
cgccccacggcaccggtcccctaatcagggcataataaat

SEQ ID NO: 15 - DNA, 40 bp, Artificial
  central sequence
cccccctccggcatccccccctaatcagtggcagtccacg

SEQ ID NO: 16 - DNA, 18 bp, Artificial
  consensus moiety
cacggtatagtctcgcca

SEQ ID NO: 17 - DNA, 23 bp, Artificial
  consensus moiety
aggggctggggtgtggttctggc

SEQ ID NO: 18 - DNA, 15 bp, Artificial
  consensus moiety
cccctaatcagtggc

SEQ ID NO: 19 - DNA, 18 bp, Artificial
  primer
gggtcaatgccaggtctc

SEQ ID NO: 20 - DNA, 18 bp, Artificial
  primer
atcggctcgcaagcagtc

SEQ ID NO: 21 - DNA, 47 bp, Artificial
  core sequence
ccaggtctccccagcctcatctcacggcatagtctcgccacactgga

SEQ ID NO: 22 - DNA, 40 bp, Artificial; identical to SEQ ID NO: 7
  core sequence

SEQ ID NO: 23 - DNA, 47 bp, Artificial
  core sequence
tcaatgccaggtctccacggtatagtctcgcccagtgccctttgttg